SEQUENCE LISTING (86 sequences)

SEQ ID NO: 1 - DNA, 21 bp, Homo sapiens
cacctttctgtgagaacggaa

SEQ ID NO: 2 - DNA, 21 bp, Homo sapiens
tagccatttgtccttgaggaa

SEQ ID NO: 3 - DNA, 19 bp, Mus musculus
gaaagctgcaagcctatct

SEQ ID NO: 4 - DNA, 21 bp, Homo sapiens
caccgttgcccaagtgaatta

SEQ ID NO: 5 - DNA, 20 bp, Artificial Sequence
  Synthetic Construct
cgccaaatacttggtcgtct

SEQ ID NO: 6 - DNA, 20 bp, Artificial Sequence
  Synthetic Construct
ccctttctgaagcagtgtcc

SEQ ID NO: 7 - DNA, 20 bp, Artificial Sequence
  Synthetic Construct
ctcttcctgcaatccgagag

SEQ ID NO: 8 - DNA, 20 bp, Artificial Sequence
  Synthetic Construct
ggatgtactgcatcgtgtcg

SEQ ID NO: 9 - DNA, 20 bp, Artificial Sequence
  Synthetic Construct
cagtagtcgctgttggatcg

SEQ ID NO: 10 - DNA, 20 bp, Artificial Sequence
  Synthetic Construct
tgcctccatattctgggaga

SEQ ID NO: 11 - DNA, 20 bp, Artificial Sequence
  Synthetic Construct
tgacccaacaaaggttgtga

SEQ ID NO: 12 - DNA, 20 bp, Artificial Sequence
  Synthetic Construct
cataccacctcccattccac

SEQ ID NO: 13 - DNA, 20 bp, Artificial Sequence
  Synthetic Construct
cgaaatggcagaagaagagg

SEQ ID NO: 14 - DNA, 20 bp, Artificial Sequence
  Synthetic Construct
tgcatgcactacagagcaca

SEQ ID NO: 15 - DNA, 19 bp, Artificial Sequence
  Synthetic Construct
tcatcggacgcacttggaa

SEQ ID NO: 16 - DNA, 21 bp, Artificial Sequence
  Synthetic Construct
caaccaccttgaatggcaaga

SEQ ID NO: 17 - DNA, 20 bp, Artificial Sequence
  Synthetic Construct
ctgcccaattccagaatcat

SEQ ID NO: 18 - DNA, 20 bp, Artificial Sequence
  Synthetic Construct
tgtaggctctgcttggtgtg

SEQ ID NO: 19 - DNA, 20 bp, Artificial Sequence
  Synthetic Construct
ccaaaggtggcattatgctt

SEQ ID NO: 20 - DNA, 20 bp, Artificial Sequence
  Synthetic Construct
tgacaggctcaatctctcca

SEQ ID NO: 21 - DNA, 20 bp, Artificial Sequence
  Synthetic Construct
acctgtgacaacccctgaag

SEQ ID NO: 22 - DNA, 20 bp, Artificial Sequence
  Synthetic Construct
tgacaccctttcttccaacc

SEQ ID NO: 23 - DNA, 20 bp, Artificial Sequence
  Synthetic Construct
gagctgaagaaggcataccg

SEQ ID NO: 24 - DNA, 20 bp, Artificial Sequence
  Synthetic Construct
cagctctcgcttctctggat

SEQ ID NO: 25 - DNA, 20 bp, Artificial Sequence
  Synthetic Construct
ggaaccaaactgaacgccta

SEQ ID NO: 26 - DNA, 20 bp, Artificial Sequence
  Synthetic Construct
atgagggcaattagcagtgg

SEQ ID NO: 27 - DNA, 20 bp, Artificial Sequence
  Synthetic Construct
tcattagggaggagccaatg

SEQ ID NO: 28 - DNA, 20 bp, Artificial Sequence
  Synthetic Construct
atcccctttcgtttttgctt

SEQ ID NO: 29 - DNA, 20 bp, Artificial Sequence
  Synthetic Construct
ccatgggaagtgtgatgatg

SEQ ID NO: 30 - DNA, 20 bp, Artificial Sequence
  Synthetic Construct
tcctactgtggctgccttct

SEQ ID NO: 31 - DNA, 20 bp, Artificial Sequence
  Synthetic Construct
acatacggggaccttccact

SEQ ID NO: 32 - DNA, 20 bp, Artificial Sequence
  Synthetic Construct
aaacatgttcttggggtcca

SEQ ID NO: 33 - DNA, 20 bp, Artificial Sequence
  Synthetic Construct
gaacatgaggaccctctgga

SEQ ID NO: 34 - DNA, 20 bp, Artificial Sequence
  Synthetic Construct
ccgctaggactttcctgttg

SEQ ID NO: 35 - DNA, 20 bp, Artificial Sequence
  Synthetic Construct
ggggaaccatgatccctagt

SEQ ID NO: 36 - DNA, 20 bp, Artificial Sequence
  Synthetic Construct
aattctccctgatcgtgtgg

SEQ ID NO: 37 - DNA, 20 bp, Artificial Sequence
  Synthetic Construct
gaggaagagtcaagggcaca

SEQ ID NO: 38 - DNA, 20 bp, Artificial Sequence
  Synthetic Construct
ctgaatccgaagctctggtc

SEQ ID NO: 39 - DNA, 20 bp, Artificial Sequence
  Synthetic Construct
atatgccttccgcatgtctc

SEQ ID NO: 40 - DNA, 20 bp, Artificial Sequence
  Synthetic Construct
gccaagggcagttcactaaa

SEQ ID NO: 41 - DNA, 19 bp, Artificial Sequence
  Synthetic Construct
aagtgttcccagtgccaca

SEQ ID NO: 42 - DNA, 20 bp, Artificial Sequence
  Synthetic Construct
gttcttattggcggctgtgt

SEQ ID NO: 43 - DNA, 20 bp, Artificial Sequence
  Synthetic Construct
aaagcccaacacacgtaagc

SEQ ID NO: 44 - DNA, 20 bp, Artificial Sequence
  Synthetic Construct
gtgcacagagacgaaggtca

SEQ ID NO: 45 - DNA, 20 bp, Artificial Sequence
  Synthetic Construct
tggcaagttgtccaaagaaa

SEQ ID NO: 46 - DNA, 20 bp, Artificial Sequence
  Synthetic Construct
acgctgggcaattcttctaa

SEQ ID NO: 47 - DNA, 20 bp, Artificial Sequence
  Synthetic Construct
ctcccttgatacagcccaag

SEQ ID NO: 48 - DNA, 20 bp, Artificial Sequence
  Synthetic Construct
tgcgcttcagggaataacat

SEQ ID NO: 49 - DNA, 20 bp, Artificial Sequence
  Synthetic Construct
agatcccggaaatccagact

SEQ ID NO: 50 - DNA, 20 bp, Artificial Sequence
  Synthetic Construct
ctacgctttgggttttccag

SEQ ID NO: 51 - DNA, 20 bp, Artificial Sequence
  Synthetic Construct
agggctgttttccatgattg

SEQ ID NO: 52 - DNA, 20 bp, Artificial Sequence
  Synthetic Construct
ccactggtccatttcttgct

SEQ ID NO: 53 - DNA, 20 bp, Artificial Sequence
  Synthetic Construct
cccaggctcagcttcgggca

SEQ ID NO: 54 - DNA, 21 bp, Artificial Sequence
  Synthetic Construct
gctcttctgtgcggacatcac

SEQ ID NO: 55 - DNA, 20 bp, Artificial Sequence
  Synthetic Construct
cacccaagaacagggtttgt

SEQ ID NO: 56 - DNA, 20 bp, Artificial Sequence
  Synthetic Construct
tggccatgggtatgttgtta

SEQ ID NO: 57 - DNA, 25 bp, Artificial Sequence
  Synthetic Construct
tgctgtctccatgtttgatgtatct

SEQ ID NO: 58 - DNA, 22 bp, Artificial Sequence
  Synthetic Construct
tctctgctccccacctctaagt

SEQ ID NO: 59 - DNA, 20 bp, Artificial Sequence
  Synthetic Construct
gccttcccccgtaaatgata

SEQ ID NO: 60 - DNA, 20 bp, Artificial Sequence
  Synthetic Construct
ttatgcgattaccgggctct

SEQ ID NO: 61 - DNA, 21 bp, Artificial Sequence
  Synthetic Construct
acgggtttcatcatgtttgtt

SEQ ID NO: 62 - DNA, 18 bp, Artificial Sequence
  Synthetic Construct
ggcagatcccctgaggtc

SEQ ID NO: 63 - DNA, 25 bp, Artificial Sequence
  Synthetic Construct
aagttctttctttttgaggaagacg

SEQ ID NO: 64 - DNA, 19 bp, Artificial Sequence
  Synthetic Construct
gaacagcggtgagtcagga

SEQ ID NO: 65 - DNA, 20 bp, Artificial Sequence
  Synthetic Construct
cgggacaggaaagagtcacc

SEQ ID NO: 66 - DNA, 20 bp, Artificial Sequence
  Synthetic Construct
cggaaaacagaggctcggaa

SEQ ID NO: 67 - DNA, 20 bp, Artificial Sequence
  Synthetic Construct
tgaacaggaagccacaacca

SEQ ID NO: 68 - DNA, 20 bp, Artificial Sequence
  Synthetic Construct
gaggccgctgggaattgtag

SEQ ID NO: 69 - DNA, 21 bp, Artificial Sequence
  Synthetic Construct
ccgtaatacttggctctcgaa

SEQ ID NO: 70 - DNA, 23 bp, Artificial Sequence
  Synthetic Construct
gaatagacctgcatgaagtgagg

SEQ ID NO: 71 - DNA, 20 bp, Artificial Sequence
  Synthetic Construct
cagcgactactgttgcttgc

SEQ ID NO: 72 - DNA, 20 bp, Artificial Sequence
  Synthetic Construct
acaggcaggacaagcgttta

SEQ ID NO: 73 - DNA, 18 bp, Artificial Sequence
  Synthetic Construct
cctagtccacccggctct

SEQ ID NO: 74 - DNA, 20 bp, Artificial Sequence
  Synthetic Construct
gaggaacctgcgagacctaa

SEQ ID NO: 75 - DNA, 20 bp, Artificial Sequence
  Synthetic Construct
acggtccactcacaatcctc

SEQ ID NO: 76 - DNA, 20 bp, Artificial Sequence
  Synthetic Construct
cccacgtggaacattttctg

SEQ ID NO: 77 - DNA, 20 bp, Artificial Sequence
  Synthetic Construct
ccgtacaccctaacatgctc

SEQ ID NO: 78 - DNA, 18 bp, Artificial Sequence
  Synthetic Construct
tggcacaacgaacactcc

SEQ ID NO: 79 - DNA, 20 bp, Artificial Sequence
  Synthetic Construct
tcttctggggaaagactgga

SEQ ID NO: 80 - DNA, 19 bp, Artificial Sequence
  Synthetic Construct
tgagttcctgcggtcaaag

SEQ ID NO: 81 - DNA, 20 bp, Artificial Sequence
  Synthetic Construct
cgagttcaggagcacaactg

SEQ ID NO: 82 - DNA, 19 bp, Artificial Sequence
  Synthetic Construct
gtcagcccctgcgatactt

SEQ ID NO: 83 - DNA, 20 bp, Artificial Sequence
  Synthetic Construct
atgtggcccggaatattggt

SEQ ID NO: 84 - DNA, 19 bp, Artificial Sequence
  Synthetic Construct
caggccgttctggagtgtc

SEQ ID NO: 85 - DNA, 19 bp, Artificial Sequence
  Synthetic Construct
agaggaggaaggcccactc

SEQ ID NO: 86 - DNA, 20 bp, Artificial Sequence
  Synthetic Construct
ctgcactctgtccctcactc

(19) United States
(12) Patent Application Publication
(10) Pub. No.: US 2018/0051253 A1
(43) Pub. Date: Feb. 22, 2018

(54) METHODS OF PREVENTING AND REVERSING STEM CELL AGING

(71) Applicant: The Regents of the University of California, Oakland, CA (US)

(72) Inventor: Danica CHEN, San Francisco, CA (US)

(73) Assignee: The Regents of the University of California, Oakland, CA (US)

(21) Appl. No.: 15/557,233

(22) PCT Filed: Mar. 18, 2016
(86) PCT No.: PCT/US2016/023270

§ 371 Date: Sep. 11, 2017

Related U.S. Application Data
(60) Provisional application No. 62/151,684, filed on Apr. 23, 2015.
(60) Provisional application No. 62/135,134, filed on Mar. 18, 2015.

(51) Int. Cl. C12N 5/0789

(52) U.S. Cl. CPC C12N 5/0647; C12N 2501/999; C12N 2510/00; C12N 2501/73

(57) ABSTRACT

Methods of preventing and reversing stem cell aging including the step of activating the mitochondrial unfolded protein response in a stem cell are described. Further described are methods of promoting stem cell maintenance and methods of preventing and/or reversing tissue degeneration or injury including the step of activating the mitochondrial unfolded protein response.

### CROSS-REFERENCE TO RELATED APPLICATIONS

This application is a U.S. National Phase patent application of PCT/US2016/023270, filed Mar. 18, 2016, which claims priority to and the benefit of U.S. Provisional Application No. 62/135,134, filed Mar. 18, 2015, and U.S. Provisional Application No. 62/151,684, filed Apr. 23, 2015, each of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 416272010500SeqList.txt, date recorded Aug. 17, 2017, size 16 KB).

### FIELD

The present disclosure relates to methods of preventing and/or reversing aging of stem cells, methods for promoting stem cell maintenance, and methods for preventing and/or reversing tissue degeneration or injury.

### BACKGROUND

Despite its central role in the life cycle of a human being, aging remains one of the least understood processes of human biology. Aging is characterized by physiological decline and increased susceptibility to pathologies and mortality. The rate of aging is controlled by evolutionarily conserved genetic pathways (1, 2). The general cause of aging is thought to be the chronic accumulation of cellular damage (2, 3).

One aspect of aging involves a diminished capacity to maintain tissue homeostasis or to repair tissues after injury. This diminished capacity is evident in certain conditions that occur with aging, such as anemia, sarcopenia (loss of muscle mass), and osteoporosis. Deterioration or aging of adult stem cells accounts for much of aging-associated compromised tissue maintenance. Adult stem cells mostly reside in a metabolically inactive quiescent state to preserve their integrity, but convert to a metabolically active proliferative state to replenish the tissue (4-6). The signals that trigger stem cells to exit the cell cycle and re-enter quiescence, and the signal transduction leading to the transition remain elusive.

By 2050 the number of people in the world that will be aged 65 or older is expected to nearly triple to about 1.5 billion, representing 16% of the world's population (NIH, National Institute on Aging, 2011). Yet in spite of this approaching burden on the world's healthcare systems, a basic understanding of the mechanisms of aging and basic treatments to combat aging are lacking. Methods are needed to alleviate aging, and in particular, methods are needed to treat the tissue degeneration and lack of tissue repair that accompanies aging.

Citation of the above documents and studies is not intended as an admission that any of the foregoing is pertinent prior art. All statements as to the contents of these documents are based on the information available to the applicants and do not constitute any admission as to the correctness of the contents of these documents.

### BRIEF SUMMARY

Accordingly, there is a need for methods to prevent or reverse aging of stem cells. The present disclosure provides methods of reversing aging of stem cells, methods of preventing aging of stem cells, methods of promoting stem cell maintenance, and methods of preventing and/or reversing tissue degeneration or injury, where the methods include a step of activating the mitochondrial unfolded protein response. The teachings herein demonstrate the surprising result that the mitochondrial unfolded protein response plays a critical role in aging of stem cells. Previously, aging of stem cells was thought to be caused primarily by an accumulation of cellular damage.

Accordingly, certain aspects of the present disclosure relate to a method of reversing aging of stem cells including a step of activating the mitochondrial unfolded protein response in a stem cell, where aging of the stem cell is reversed. Other aspects of the present disclosure relate to a method of preventing aging of stem cells including a step of activating the mitochondrial unfolded protein response in a stem cell, where aging of the stem cell is prevented. Other aspects of the present disclosure relate to a method of promoting stem cell maintenance including a step of activating the mitochondrial unfolded protein response in a stem cell, where the stem cell continues to self-renew. Other aspects of the present disclosure relate to a method of preventing and/or reversing tissue degeneration or injury including a step of activating the mitochondrial unfolded protein response in a stem cell, where the stem cell is in an animal and where degeneration or injury of a tissue in the animal is prevented and/or reversed.

In some embodiments of any of the above aspects, the mitochondrial unfolded protein response is activated by activating SIRT7 in the stem cell. In some embodiments the mitochondrial unfolded protein response is activated by activating a mitochondrial stress protein. In some embodiments the mitochondrial stress protein is selected from the group consisting of mtDnaJ, HSP60, HSP10, and ClpP in the stem cell.

In certain embodiments where the mitochondrial unfolded protein response is activated by activating SIRT7 in the stem cell, SIRT7 is activated by increasing the transcription of the sirt7 gene in the stem cell. In some embodiments SIRT7 is activated by increasing the translation of SIRT7 protein in the stem cell. In some embodiments SIRT7 is activated by delivering an exogenous copy of the sirt7 gene to the stem cell wherein the exogenous sirt7 gene is expressed in the stem cell. In some embodiments the exogenous copy of sirt7 gene is delivered to the stem cell with a viral vector. In some embodiments SIRT7 is activated by a small molecule. In some embodiments SIRT7 is activated by increasing intracellular NAD levels. In some embodiments intracellular NAD levels are increased by delivering small molecules that activate NAD synthesis enzymes to the stem cell. In some embodiments intracellular NAD levels are increased by increasing the level of a NAD precursor in the stem cell. In some embodiments the NAD precursor is selected from the group consisting of nicotinamide mono nucleotide (NMN) and nicotinamide riboside (NR). In some embodiments intracellular NAD levels are increased by increasing the level of a NAD biosynthesis enzyme in the stem cell.

In some embodiments of aspects of the present disclosure related to a method of reversing aging of stem cells, the stem cell exhibits one or more of the characteristics selected from the group consisting of reduced occurrence of cell death, increased quiescence, increased occurrence of self-renewal, increased ability to repopulate its organ or tissue of origin, increased homing ability, and a change in differentiation profile.

In some embodiments of any of the aspects of the present disclosure, the stem cell is in an animal. In certain embodiments the animal is a human. In certain embodiments the stem cell is a hematopoietic stem cell. In some embodiments the stem cell originated in an organ or tissue selected from the group consisting of brain, spinal cord, peripheral blood, blood vessels, skeletal muscle, skin, teeth, hair follicle, heart, gut, liver, ovarian epithelium, and testis. In embodiments where the stem cell is a hematopoietic stem cell, the hematopoietic stem cell exhibits improved performance in an assay selected from the group consisting of HSC engraftment, bone marrow reconstitution, and competitive transplantation. In embodiments where the stem cell is in an animal and the animal is a human, blood drawn from the human does not exhibit one or more characteristics selected from the group consisting of increased myeloid differentiation, fewer lymphoid cells, and anemia. In some embodiments the stem cell is an aging stem cell.

In some embodiments of aspects of the disclosure related to a method of preventing aging of stem cells, aging of the stem cell is delayed.

### BRIEF DESCRIPTION OF THE DRAWINGS

FIGS. 1A-1D depict the stabilization of SIRT7 by NRF1 at the promoters of mitochondrial translational machinery components. FIG. 1A depicts co-immunoprecipitation of transfected Flag-tagged SIRT7 with endogenous NRF1 from 293T cells. FIG. 1B depicts co-immunoprecipitation of transfected endogenous SIRT7 with endogenous NRF1 from 293T cells. FIG. 1C depicts chromatin immunoprecipitation followed by quantitative real-time PCR (ChIP-qPCR) showing SIRT7 occupancy at gene promoters. FIG. 1D depicts chromatin immunoprecipitation followed by quantitative real-time PCR (ChIP-qPCR) showing NRF1 occupancy at gene promoters. Error bars represent standard error (SE). **: p<0.01. ***: p<0.001. ns: p>0.05. Student's t test.

FIGS. 2A-2C depict co-occupation by SIRT7 and NRF1 of the same genomic regions at the promoters of mitochondrial translation machinery components. FIG. 2A depicts SIRT7 bound to the promoters of mRPs but not other mitochondrial genes. FIG. 2B depicts NRF1 bound to the promoters of mRPs and other mitochondrial genes, but not NME1, a known target of SIRT7. SIRT7 and NRF1 occupancy at gene promoters in 293T cells was determined by ChIP-PCR compared to IgG negative control samples. All samples were normalized to input DNA. FIG. 2C depicts a schematic representation of NRF1 and NRF2 consensus binding sequences, and SIRT7 binding sites at gene promoters. SIRT7 binding sites were determined in a ChIP-seq study (Barber et al. Nature 2012). Error bars represent SE. **: p<0.01. ***: p<0.001.

FIGS. 3A-3B depict knockdown of NRF1 with siRNA in cells. FIG. 3A depicts an immunoblot showing reduced expression of NRF1 protein in 293T cells that were transfected with NRF1 siRNA. FIG. 3B depicts results of qPCR showing a 40% reduction in NRF1 expression in 293T cells that were transfected with NRF1 siRNA. Error bars represent SE. *: p<0.05.

FIGS. 4A-4J depict limitation of mitochondrial activity, proliferation, and PFS^{mt} by SIRT7. FIG. 4A depicts increased expression of GFM2 and MRPL24 in SIRT7 KD cells. FIG. 4B depicts the abrogation of increased expression of GFM2 and MRPL24 in SIRT7 KD cells by NRF1 siRNA. FIG. 4C depicts increased mitochondrial mass in SIRT7 KD cells determined by MTG staining. n=3. FIG. 4D depicts increased mitochondrial mass in SIRT7 KD cells determined by mitochondrial DNA quantification. n=3. FIG. 4E depicts increased proliferation in SIT7 KD cells. n=3. FIG. 4F depicts SIRT7 expression induced by PFS^{mt}. Dox: doxycycline. EB: ethidium bromide. FIG. 4G depicts increased accumulation of misfolded ΔOTC in SIRT7 KD cells and rescue by NRF1 siRNA. OTC was used as a control. FIG. 4H depicts increased apoptosis in SIRT7 KD cells treated with ethidium bromide. n=3. FIG. 4I depicts abrogation of increased PFS^{mt} in SIRT7 KD cells by NRF1 siRNA. FIG. 4J depicts a proposed model of SIRT7's role in mediating the UPR^{mt}. Error bars represent SE. *: p<0.05. **: p<0.01. Student's t test.

FIGS. 5A-5B depict repression of NRF1 transcription by SIRT7. FIG. 5A depicts repression of the expression of mRPs by SIRT7. SIRT7 was knocked down in 293T cells via shRNA. Gene expression in SIRT7 KD cells and control cells was determined by qPCR. FIG. 5B depicts the repression of the expression of mRPs by SIRT7 via NRF1. SIRT7 KD 293T cells and control cells were treated with control or NRF1 siRNA as indicated. Gene expression was determined by qPCR. NRF1 KD abrogated SIRT7-mediated transcriptional repression of mRPs in cells. Error bars represent SE **: p<0.01. ***: p<0.001. ns: p>0.05.

FIGS. 6A-6J depict the limitation of mitochondrial activity and of cell proliferation by SIRT7. FIGS. 6A-6D depict increased mitochondrial activity in SIRT7 KD cells. FIG. 6A depicts a comparison of SIRT7 KD 293T cells and control cells for citrate synthase activity quantification, FIG. 6B depicts cellular ATP quantification using a luminescent assay, and FIGS. 6C and 6D depict Seahorse analyses. FIGS. 6E-6G depict OCR (oxygen consumption rate) and ECAR (extracellular acidification rate). Overexpression of WT but not a catalytically inactive mutant (HY) SIRT7 reduced mitochondrial activity and proliferation. FIG. 6E depicts a comparison of 293T cells overexpressing WT and mutant SIRT7 and control cells for MTG staining. FIG. 6F depicts a comparison of 293T cells overexpressing WT and mutant SIRT7 and control cells for Seahorse analyses. FIG. 6G depicts a comparison of 293T cells overexpressing WT and mutant SIRT7 and control cells for cell proliferation analyses. Cells were counted using a Vi-Cell analyzer. FIGS. 6H-6J depict restoration of increased mitochondrial activity and proliferation in SIRT7 KD cells by NRF1 siRNA. FIG. 6H depicts SIRT7 KD 293T cells and control cells treated with control or NRF1 siRNA and analyzed for MTG staining. FIG. 6I depicts cellular ATP quantification. FIG. 6J depicts cell proliferation analyses. Error bars represent SE *: p<0.05. **: p<0.01. ***: p<0.001. ns: p>0.05.

FIGS. 7A-7F depict that SIRT7 promotes nutritional stress resistance. FIG. 7A depicts that SIRT7 expression is increased upon glucose starvation. qPCR is shown comparing SIRT7 expression in 293T cells growing in medium containing 25 mM glucose and without glucose. FIGS. 7B-7D depict SIRT7 causing an increase in nutrient starvation stress resistance. FIG. 7B depicts SIRT7 OE in 293T cells and control cells that were deprived of glucose for 68 hours. FIGS. 7C and 7D depict SIRT7 KD 293T cells and control cells that were deprived of glucose (FIG. 7C) and glutamine (FIG. 7D) for 48 hours. Cells were counted using a Vi-Cell analyzer. FIGS. 7E and 7F depict that NRF1 KD attenuated the sensitivity of SIRT7 deficient cells to glucose (FIG. 7E) or glutamine (FIG. 7F) starvation. SIRT7 KD 293T cells and control cells were treated with control or NRF1 siRNA. Cells were deprived of glucose or glutamine for 48 hours. Cells were counted using a Vi-Cell analyzer. Error bars represent SE *: p<0.05. **: p<0.01.

FIGS. 8A-8C depict that SIRT7 represses NRF1 activity to suppress PFS^{mt}. FIGS. 8A and 8B depict SIRT7 repression of PFS^{mt}. SIRT7 KD 293T cells and control cells were treated with or without EB for 7 days. The expression of UPR^{mt} genes (ClpP and HSP60) were determined by immunoblots (FIG. 8A) or qPCR (FIG. 8B). FIG. 8C depicts SIRT7 repression of NRF1 activity to suppress PFS^{mt}. SIRT7 KD 293T cells and control cells were treated with control or NRF1 siRNA. The expression of UPR^{mt} (ClpP, HSP10, HSP60, mtDnaJ) and UPR^{ER} genes (Grp78) was determined by qPCR.

FIGS. 9A-9C depict SIRT7 expression in various tissues and cellular compartments. FIG. 9A depicts that SIRT7 is highly expressed in the bone marrow. The expression of SIRT7 in various tissues was compared by qPCR. FIG. 9B depicts that SIRT7 is ubiquitously expressed in various hematopoietic cellular compartments in the bone marrow. Various cell populations in the bone marrow were isolated via cell sorting based on cell surface markers. HSC, Lin−c-Kit+Sca1+CD150+CD48−; multipotent progenitors (MPPs), Lin−c−Kit+Sca1+CD150−CD48−; CD48+, Lin−c-Kit+Sca1+CD48+; myeloid progenitors (MPs), Lin−c−Kit+Sca1−; and differentiated blood cells, Lin+. The expression of SIRT7 was determined by qPCR. FIG. 9C depicts the gating strategy for sorting HSCs. Error bars represent SE.

FIGS. 10A-10I depict that SIRT7 ensures HSC maintenance. FIG. 10A depicts qPCR showing increased PFS^{mt} in SIRT7^{-/-} HSCs. n=4. FIGS. 10B and 10C depict MTG staining and mitochondrial DNA quantification showing increased mitochondrial mass in SIRT7^{-/-} HSCs. n=4. MP: myeloid progenitor cells. Lin−: lineage negative cells. FIG. 10D depicts in vivo BrdU incorporation showing increased proliferation of SIRT7^{-/-} HSCs. n=4. FIG. 10E depicts increased propensity of SIRT7^{-/-} HSCs to cycle upon ex vivo culture with cytokines, indicated by BrdU pulse. n=4. FIG. 10F depicts that mice reconstituted with SIRT7^{-/-} BMCs have increased sensitivity to 5-Fluorouracil. n=12. Log-rank test. FIG. 10G depicts competitive transplantation using SIRT7^{+/+} and SIRT7^{-/-} BMCs as donors, which shows the reduced reconstitution capacity of SIRT7^{-/-} HSCs. n=15. Representative of 5 transplants. FIG. 10H depicts reduced white blood cell count in SIRT7^{-/-} mice. FIG. 10I depicts myeloid-biased differentiation in the peripheral blood (PB) of SIRT7^{-/-} mice. MNCs: mononuclear cells. n=7. Error bars represent SE. *: p<0.05. ***: p<0.001. ns: p>0.05. Student's t test.

FIG. 11 depicts that SIRT7 suppresses mitochondrial number in HSCs. Electron microscopy of SIRT7^{+/+} and SIRT7^{-/-} HSCs is depicted.

FIGS. 12A-12G depict that SIRT7 ensures HSC maintenance. FIG. 12A depicts competitive transplantation using HSCs isolated from SIRT7^{+/+} and SIRT7^{-/-} mice as donors, which shows reduced reconstitution capacity of SIRT7^{-/-} HSCs. n=15. FIG. 12B depicts the frequency of HSCs in the bone marrow of SIRT7^{+/+} and SIRT7^{-/-} mice, as determined via flow cytometry. n=4. FIG. 12C depicts SIRT7^{+/+} or SIRT7^{-/-} HSCs transduced with NRF1 KD lentivirus or control lentivirus that were used as donors in a competitive transplantation assay. Data shown are the frequency of donor-derived HSCs in the bone marrow of recipient mice. n=6. FIG. 12D depicts Annexin V staining showing increased apoptosis in SIRT7^{-/-} HSCs under transplantation stress. n=7. FIG. 12E depicts competitive transplantation using HSCs isolated from SIRT7^{+/+} and SIRT7^{-/-} mice as donors. Data shown are the distribution of donor-derived myeloid or lymphoid lineage in the peripheral blood of transplant recipients. n=15. FIGS. 12F and 12G depict SIRT7^{+/+} or SIRT7^{-/-} HSCs transduced with SIRT7 lentivirus or control lentivirus that were used as donors in a competitive transplantation assay. Data shown are the percentage of total donor-derived contribution (FIG. 12F) and donor-derived mature hematopoietic subpopulations (FIG. 12G) in the peripheral blood of recipients. n=7. Error bars represent SE. *: p<0.05. **: p<0.01. ***: P<0.001. ns: p>0.05.

FIGS. 13A-13D depict repression of NRF1 activity by SIRT7 to ensure HSC maintenance. SIRT7^{+/+} or SIRT7^{-/-} HSCs transduced with NRF1 KD lentivirus or control lentivirus were used as donors in a competitive transplantation assay. Data shown are qPCR analyses of UPR^{mt} gene expression (FIG. 13A) and cell cycle analysis with Ki67 staining (FIG. 13B) of donor-derived HSCs, the percentage of total donor-derived contribution (FIG. 13C) and donor-derived mature hematopoietic subpopulations (FIG. 13D) in the peripheral blood of recipients. n=7. Error bars represent SE. *: p<0.05. **: p<0.01.

FIGS. 14A-14E depict that HSC aging is regulated by SIRT7. FIG. 14A depicts qPCR showing reduced SIRT7 expression in aged HSCs. n=3. FIGS. 14B and 14C depict qPCR showing that increased PFS^{mt} in aged HSCs is rescued by SIRT7 overexpression. n=3. FIGS. 14D and 14E depict competitive transplantation using aged HSCs transduced with SIRT7 or control lentivirus as donors. SIRT7 overexpression increased reconstitution capacity and reversed myeloid-biased differentiation of aged HSCs. n=7. Error bars represent SE. *: p<0.05. **: p<0.01. Student's t test.

FIGS. 15A-B depict HSC aging regulated by NRF1. FIGS. 15A and 15B depict competitive transplantation using aged HSCs transduced with NRF1 KD virus or control virus as donors, which shows that NRF1 inactivation increased reconstitution capacity and reversed myeloid-biased differentiation of aged HSCs. Data shown are the percentage of donor-derived cells in the peripheral blood of the recipients (FIG. 15A) and the percentage of lymphoid and myeloid cells in donor-derived cells in the peripheral blood of the recipients (FIG. 15B). n=7. Error bars represent SE *: p<0.05.

### DETAILED DESCRIPTION

In one aspect, the present disclosure provides methods of reversing aging of stem cells where the method includes the step of activating the mitochondrial unfolded protein response in a stem cell and where aging of the stem cell is reversed. In another aspect, the present disclosure provides methods of preventing aging of stem cells where the method includes the step of activating the mitochondrial unfolded protein response in a stem cell and where aging of the stem cell is prevented. In another aspect, the present disclosure provides methods of promoting stem cell maintenance where the method includes the step of activating the mitochondrial unfolded protein response in a stem cell and where the stem cell continues to self-renew. In another aspect, the present disclosure provides methods of preventing and/or reversing tissue degeneration or injury where the method includes the step of activating the mitochondrial unfolded protein response in a stem cell, where the stem cell is in an animal, and where degeneration or injury of a tissue in the animal is prevented and/or reversed.

### Activating the Mitochondrial Unfolded Protein Response

The methods of the present disclosure include a step of activating the mitochondrial unfolded protein response in a stem cell. The mitochondrial unfolded protein response is a retrograde signaling pathway leading to transcriptional upregulation of mitochondrial chaperones and stress relief. Perturbation of mitochondrial proteostasis, a form of mitochondrial stress, is one way by which the mitochondrial unfolded protein response is activated.

Activating SIRT7 in the Stem Cell

In some embodiments, the mitochondrial unfolded protein response is activated by activating SIRT7 in the stem cell. SIRT7 may be activated in the stem cell by any methods known in the art. For example, in some embodiments, SIRT7 is activated by increasing the transcription of the sirt7 gene in the stem cell. In other embodiments, SIRT7 is activated by increasing the translation of SIRT7 protein in the stem cell. Methods of increasing the transcription of the sirt7 gene or of increasing the translation of SIRT7 protein in the stem cell include, without limitation, increasing the gene copy number of sirt7 in the stem cell and expressing sirt7 with a promoter that gives higher levels of expression of sirt7 than that of its native promoter. In some embodiments, SIRT7 is activated by delivering an exogenous copy of the sirt7 gene to the stem cell such that the exogenous sirt7 gene is expressed in the stem cell. In certain embodiments, the exogenous copy of the sirt7 gene is delivered to the stem cell with a viral vector. The viral vector may be, for example, derived from a lentivirus.

In some embodiments SIRT7 is activated by a small molecule. A small molecule may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively the compound may be an inorganic compound. Inorganic compounds are derived from mineral sources and include all compounds without carbon atoms (except carbon dioxide, carbon monoxide and carbonates). Small molecule activators of SIRT7 may be identified, for example, by high throughput screening, such as a forward chemical genetic screen. Small molecules that activate SIRT7 may be delivered to stem cells by any method known in the art. For example, small molecule activators of SIRT7 may be delivered to stem cells with nanoparticles that are targeted to a particular type of stem cell. See, e.g., WO2007133750 A2 and WO2006033679 A2.

SIRT7 activity is dependent on NAD. Accordingly, in some embodiments SIRT7 is activated by increasing intracellular NAD levels. In some embodiments intracellular NAD levels are increased by delivering small molecules to the stem cell that activate NAD biosynthesis enzymes. NAD biosynthesis enzymes include nicotinamide phosphoribosyltransferase (NamPRT), nicotinic acid phosphoribosyltransferase (NAPRT), quinolinic acid phosphoribosyltransferase (QAPRT), nicotinamide riboside kinase (NRK), nicotinamide adenylyltransferase (NMNAT), and NAD synthetase (NADS). Small molecules that activate NAD biosynthesis enzymes may be delivered to stem cells by any method known in the art. For example, small molecule activators of NAD biosynthesis enzymes may be delivered to stem cells with nanoparticles that are targeted to a particular type of stem cell. See, e.g., WO2007133750 A2 and WO2006033679 A2.

In other embodiments intracellular NAD levels are increased by increasing the level of a NAD precursor in the stem cell. The NAD precursor may be, for example, nicotinamide mono nucleotide (NMN), nicotinamide riboside (NR), or nicotinamide. The level of NAD precursors in the stem cell may be increased by any method known in the art. For example, NAD precursors may be ingested as nutritional supplements.

In some embodiments, intracellular NAD levels are increased by increasing the level of a NAD biosynthesis enzyme in the stem cell. Increasing the level of a NAD biosynthesis enzyme in the stem cell may be achieved, for example, by increasing the transcription of the gene encoding such an enzyme or by increasing the translation of the enzyme in the stem cell. An exogenous copy of a gene encoding a NAD biosynthesis enzyme may also be delivered to the stem cell such that the exogenous gene is expressed in the stem cell. The exogenous copy of the gene encoding a NAD biosynthesis enzyme may be delivered to the stem cell with a viral vector such as a vector derived from a lentivirus.

Activating a Mitochondrial Stress Protein

In some embodiments, the mitochondrial unfolded protein response is activated by activating a mitochondrial stress protein in the stem cell. Mitochondrial stress proteins are encoded by nuclear genes that are transcriptionally upregulated in response to the accumulation of unfolded protein within the mitochondrial matrix (Zhao et al., EMBO J 2002, 21(17): 4411-4419). Mitochondrial stress proteins include, without limitation, mtDnaJ, HSP60, HSP10, and ClpP. Mitochondrial stress proteins may be activated, for example, by increasing the level of a mitochondrial stress protein in the stem cell. Increasing the level of a mitochondrial stress protein in the stem cell may be achieved, for example, by increasing the transcription of the gene encoding such a protein or by increasing the translation of such a protein in the stem cell. An exogenous copy of a gene encoding a mitochondrial stress protein may also be delivered to the stem cell such that the exogenous gene is expressed in the stem cell. The exogenous copy of the gene encoding a mitochondrial stress protein may be delivered to the stem cell with a viral vector such as a vector derived from a lentivirus.

### Methods of Reversing Aging of Stem Cells

In one aspect of the present disclosure, a method of reversing stem cell aging where the method includes the step of activating the mitochondrial unfolded protein response in a stem cell and where aging of the stem cell is reversed is provided. The aging of a stem cell is reversed where any property of an aging stem cell changes to be characteristic of a non-aging stem cell. Aging stem cells may exhibit one or more of the following properties or characteristics: decreased per cell repopulating activity, decreased self-renewal and homing abilities, myeloid skewing of differentiation, and increased apoptosis with stress (Janzen et al., Nature, 2006, v. 443, p. 421). Thus, in some embodiments, aging of the stem cell is reversed where the stem cell exhibits one or more of the following characteristics: reduced occurrence of cell death, increased quiescence, increased occurrence of self-renewal, increased ability to repopulate its organ or tissue of origin, increased homing ability, a change in differentiation profile, decreased transcriptional activity, decreased cell size, decreased mitochondrial metabolic activity, and a change in chromatin profile. Any methods known in the art to evaluate these properties may be used to identify whether the aging of a stem cell is reversed. For example, assays such as competitive repopulation and transplantation, engraftment, colony-forming unit assays, long-term culture assays, cell proliferation assays such as BrdU or EdU incorporation, transcriptional profiling by microarray analysis, chromatin immunoprecipitation sequencing (ChIP-seq), cellular ATP measurements, secondary neurophore formation assays, secondary neurophore differentiation assays, and caspase activity assays may be used. Description of these assays and others may be found, for example, in the following publications: Mohrin et al., Science 347, 1374 (Mar. 20, 2015); Tang, Rando, EMBO J. 33, 2782 (Dec. 1, 2014); Rodgers et al., Nature 510, 393 (Jun. 19, 2014); Liu, et al., Cell Rep. 4, 189 (Jul. 11, 2013); Renault et al., Cell Stem Cell. 5, 527 (Nov. 6, 2009); Webb et al., Cell Rep. 4, 477 (Aug. 15, 2013); Keyes et al., PNAS 110, E4950 (Dec. 17, 2013); Hsu et al., Nat. Med. 20, 847 (August 2014).

In some embodiments where the stem cell is a hematopoietic stem cell, aging of the stem cell is reversed where the hematopoietic stem cell exhibits improved performance in an assay such as HSC engraftment, bone marrow reconstitution, or competitive transplantation. These assays measure the ability of transplanted stem cells to gain access to the bone marrow of an irradiated recipient animal, take up residence in the bone marrow, undergo self-renewing cell division to produce a larger pool of hematopoietic stem cells, and differentiate to generate different cell types (See, e.g., J. Clin Invest. 2002; 110(3): 303-304). In some embodiments where the stem cell is in a human and where the stem cell is a hematopoietic stem cell, reversal of stem cell aging is detected by drawing and testing blood. Blood drawn from a human where stem cell aging has been reversed does not exhibit one or more of the following characteristics typical of aging, such as increased myeloid differentiation, fewer lymphoid cells, or anemia. Myeloid differentiation, lymphoid cells, and anemia may be measured with standard assays known in the art (See, e.g., Pang et al., PNAS 108, 20012 (Dec. 13, 2011).

### Methods of Preventing Aging of Stem Cells

In one aspect of the present disclosure, a method of preventing stem cell aging where the method includes a step of activating the mitochondrial unfolded protein response in a stem cell and where aging of the stem cell is prevented is provided. In some embodiments, aging of a stem cell is prevented where aging of the stem cell is delayed. Aging of a stem cell is delayed where, for example, characteristics or properties of an aging stem cell occur later than those characteristics or properties would occur for an unmanipulated stem cell. A delay in aging of a stem cell may be apparent when stem cells from an older adult have similar characteristics to stem cells from a young adult. Characteristics or properties of an aging stem cell and of stem cells from an older adult include, without limitation, decreased per cell repopulating activity, decreased self-renewal and homing abilities, myeloid skewing of differentiation, and increased apoptosis with stress.

### Methods of Promoting Stem Cell Maintenance

In one aspect of the present disclosure, a method of promoting stem cell maintenance where the method includes the step of activating the mitochondrial unfolded protein response in a stem cell and where the stem cell continues to self-renew is provided. Self-renewal of stem cells is division of the stem cell with maintenance of the undifferentiated state. Assays to measure the self-renewal capabilities of stem cells include, without limitation, a serial dilution assay, a colony-forming unit assay, a long-term culture assay, and a secondary neurophore formation assay.

Methods of Preventing and/or Reversing Tissue Degeneration or Injury

Deterioration or aging of adult stem cells accounts for much of aging-associated compromised tissue maintenance. Certain conditions that occur with aging reflect compromised tissue maintenance. These conditions include, for example, anemia, sarcopenia, and osteoporosis. In addition, aging of adult stem cells contributes to a diminished capacity to repair tissues after injury. This diminished capacity to repair is exemplified in poor wound healing of skin, reduced angiogenesis in damaged organs, reduced remyelination in the central nervous system in response to demyelinating conditions, and fibrous scarring instead of the formation of new muscle fibers following a necrotic injury (Cell Cycle 4:3, 407-410; 2005). Accordingly, in one aspect of the present disclosure, a method of preventing and/or reversing tissue degeneration or injury where the method includes the step of activating the mitochondrial unfolded protein response in a stem cell, where the stem cell is in an animal, and where degeneration or injury of a tissue in the animal is prevented and/or reversed is provided. The degenerated or injured tissue may be, without limitation, brain, spinal cord, peripheral blood, blood vessels, skeletal muscle, skin, teeth, hair follicle, heart, gut, liver, ovarian epithelium, or testis.

### Stem Cells

The methods described herein may be performed with a stem cell from any animal and from any tissue or organ, without limitation. A stem cell is generally defined as a cell that is capable of renewing itself and can give rise to more than one type of cell through asymmetric cell division. Stem cells exist in many tissues of embryos and adult mammals. Pluripotent stem cells have the ability to differentiate into almost any cell type, and multipotent stem cells have the ability to differentiate into many cell types. In certain embodiments, the stem cell originated in brain, spinal cord, peripheral blood, blood vessels, skeletal muscle, skin, teeth, hair follicle, heart, gut, liver, ovarian epithelium, or testis. In certain embodiments of the methods of the present disclosure, the stem cell is in an animal. In some embodiments, the animal is a human.

Hematopoietic Stem Cells (HSCs)

In certain embodiments, the stem cell is a hematopoietic stem cell. Hematopoietic stem cells may be isolated from blood (i.e. hematopoietic tissue). Possible sources of human hematopoietic tissue include, but are not limited to, embryonic hematopoietic tissue, fetal hematopoietic tissue, and post-natal hematopoietic tissue. Embryonic hematopoietic tissue can be yolk sac or embryonic liver. Fetal hematopoietic tissue can come from fetal liver, fetal bone marrow and fetal peripheral blood. The post-natal hematopoietic can be cord blood, bone marrow, normal peripheral blood, mobilized peripheral blood, hepatic hematopoietic tissue, or splenic hematopoietic tissue.

HSCs suitable for use with the methods of the present disclosure may be obtained by any suitable technique known in the art. For example, HSCs may be found in the bone marrow of a donor, which includes femurs, hip, ribs, sternum, and other bones. Any method known in the art for extracting or harvesting bone marrow cells may be used. In one non-limiting example, HSCs may be obtained directly from the marrow cavity of the hip using a needle and syringe to aspirate cells from the marrow cavity. Rich marrow may be obtained from the hip by performing multiple small aspirations.

Alternatively, suitable HSCs may be obtained from peripheral blood cells found in the blood of a donor, optionally following pre-treatment with cytokines, such as G-CSF (granulocyte colony-stimulating factors), that induce HSCs to be released from the bone marrow compartment of the donor. HSCs may also be obtained from peripheral blood that has undergone an apheresis procedure to enrich for HSCs. Any apheresis procedure known in the art may be used. In certain embodiments, the apheresis procedure is a leukapheresis procedure.

Additionally, suitable HSCs may be obtained from umbilical cord blood, placenta, and mobilized peripheral blood. For experimental purposes, fetal liver, fetal spleen, and AGM (Aorta-gonad-mesonephros) of animals are also useful sources of HSCs. Additionally, HSCs may be procured from a source that obtained HSCs from the bone marrow, peripheral blood, umbilical cord, or fetal tissue of a donor.

In some embodiments, HSCs are obtained from a human umbilical cord or placenta. Another source of HSCs that may be utilized is the developing blood-producing tissues of fetal animals. In humans, HSCs may be found in the circulating blood of a human fetus by about 12 to 18 weeks.

In some embodiments, human HSCs are obtained from any source, e.g., the bone marrow, umbilical cord, peripheral blood, or fetal tissue of blood, of type A+, A−, B+, B−, O+, O−, AB+, and AB− donors. In other embodiments, human HSCs are obtained from any source, e.g., the bone marrow, umbilical cord, peripheral blood, or fetal tissue of blood, of universal donors or donors having a rare blood type.

In other embodiments, human HSCs are obtained from any source, e.g., the bone marrow, umbilical cord, peripheral blood, or fetal tissue of blood, of donors having an aging disorder or aging-associated condition that would benefit from a transplantation of HSCs and/or transfusion of blood. Such donors may also be the recipients. Advantageously, HSCs obtained from such donor may be used for personalized HSC and/or blood therapy.

In one non-limiting example, human HSCs may be obtained by anesthetizing the stem cell donor, puncturing the posterior superior iliac crest with a needle, and performing aspiration of bone marrow cells with a syringe. In another non-limiting example, HSCs may be obtained from the peripheral blood of a donor, where a few days prior to harvesting the stem cells form the peripheral blood, the donor is injected with G-CSF in order to mobilize the stem cells to the peripheral blood.

Cells obtained from, for example, bone marrow, peripheral blood, or cord blood, are typically processed after extraction or harvest. Any method known in the art for processing extracted or harvested cells may be used. Examples of processing steps include, without limitation, filtration, centrifugation, screening for hematopathologies, screening for viral and/or microbial infection, erythrocyte depletion, T-cell depletion to reduce incidence of graft-versus-host disease in allogenic stem cell transplant recipients, volume reduction, cell separation, resuspension of cells in culture medium or a buffer suitable for subsequent processing, separation of stem cells from non-stem cells (e.g., stem cell enrichment), ex vivo or in vitro stem cell expansion with growth factors, cytokines, and/or hormones, and cryopreservation.

Any suitable method for stem cell enrichment known in the art may be used. Examples of stem cell enrichment methods include, without limitation, fluorescence activated cell sorting (FACS) and magnetic activated cell sorting (MACS).

Accordingly, in certain embodiments, HSCs suitable for use in the methods of the present disclosure are human HSCs.

Identification and Targeting of HSCs

HSCs obtained from a donor may be identified and/or enriched by any suitable method of stem cell identification and enrichment known in the art, such as by utilizing certain phenotypic or genotypic markers. For example, in some embodiments, identification of HSCs includes using cell surface markers associated with HSCs or specifically associated with terminally differentiated cells of the system. Suitable surface markers may include, without limitation, one or more of c-kit, Sca-1, CD4, CD34, CD38, Thy1, CD2, CD3, CD4, CD5, CD8, CD43, CD45, CD59, CD90, CD105, CD133, CD135, ABCG2, NK1.1, B220, Ter-119, Flk-2, CDCP1, Endomucin, Gr-1, CD46, Mac-1, Thy1.1, and the signaling lymphocyte activation molecule (SLAM) family of receptors. Examples of SLAM receptors include, without limitation, CD150, CD48, and CD244.

In some embodiments of the present disclosure, small molecules are delivered to stem cells. Cell surface markers associated with HSCs, such as c-kit, Sca-1, CD4, CD34, CD38, Thy1, CD2, CD3, CD4, CD5, CD8, CD43, CD45, CD59, CD90, CD105, CD133, CD135, ABCG2, NK1.1, B220, Ter-119, Flk-2, CDCP1, Endomucin, Gr-1, CD46, Mac-1, Thy1.1, and the signaling lymphocyte activation molecule (SLAM) family of receptors, may be helpful in targeting small molecules to HSCs where the HSC is in an animal. For example, nanoparticles carrying small molecules for delivery to a stem cell may be targeted to an HSC based on its expression of cell-surface molecules.

Additionally, HSCs obtained from a donor may be separated from non-stem cells by any suitable method known in the art including, without limitation, fluorescence activated cell sorting (FACS) and magnetic activated cell sorting (MACS).

In one non-limiting example, human peripheral blood cells are incubated with antibodies recognizing c-kit, Sca-1, CD34, CD38, Thy1, CD2, CD3, CD4, CD5, CD8, CD43, CD45, CD59, CD90, CD105, CD133, ABCG2, NK1.1, B220, Ter-119, Flk-2, CDCP1, Endomucin, or Gr-1. Antibodies for CD2, CD3, CD4, CD5, CD8, NK1.1, B220, Ter-119, and Gr-1 are conjugated with magnetic beads. The cells expressing CD2, CD3, CD4, CD5, CD8, NK1.1, B220, Ter-119, or Gr-1 are retained in the column equipped to trap magnetic beads and cells attached to magnetic bead conjugated antibodies. The cells that are not captured by the MACS column are subjected to FACS analysis. Antibodies for c-kit, Sca-1, CD34, CD38, Thy1, are conjugated with fluorescent materials known in the art. The cells that are CD34⁺, CD38^{low/-} c-kit^{-/low}, Thy1⁺ are separated from the rest of sample by virtue of the types of fluorescent antibodies associated with the cells. These cells are provided as human long-term HSCs suitable for use with any of the methods of the present disclosure.

In another non-limiting example, cells obtained from a subject are labeled with the same set of magnetic bead conjugated antibodies as described above (antibodies against one or more of CD2, CD3, CD4, CD5, CD8, NK1.1, B220, Ter-119, or Gr-1) and fluorescent conjugated CD150, CD244 and/or CD48 antibodies. After removing cells captured by the magnetic bead conjugated antibodies from the sample, the sample is analyzed by FACS and CD150⁺, CD244⁻ and CD48⁻ cells are retained as long-term HSCs.

In some embodiments, HSCs utilized in the methods of the present disclosure contain one or more of the markers: c-kit⁺, Sca-1⁺, CD34^{low/-}, CD38⁺, Thy1^{+/low}, CD34⁺, CD38^{low/-}, c-kit^{-/low}, and/or Thy1⁺. In some embodiments, the HSCs utilized in the methods of the present disclosure lack one or more of the markers: CD2, CD3, CD4, CD5, CD8, NK1.1, B220, Ter-119, and/or Gr-1. In certain embodiments, the HSCs utilized in the methods of the present disclosure are of an A+, A−, B+, B−, O+, O−, AB+, or AB− type.

Alternatively, suitable HSCs may be obtained from a non-human source. Suitable non-human HSCs may be isolated from, femurs, hip, ribs, sternum, and other bones of a non-human animal, including, without limitation, laboratory/research animals, rodents, pets, livestock, farm animals, work animals, pack animals, rare or endangered species, racing animals, and zoo animals. Further examples of suitable non-human animals include, without limitation, monkeys, primates, mice, rats, guinea pigs, hamsters, dogs, cats, horses, cows, pigs, sheep, goats, and chickens. For example, HSCs may be obtained from murine bone marrow cells, by incubating the bone marrow cells with antibodies recognizing cell surface molecules such as one or more of c-kit, Sca-1, CD34, CD38, Thy1, CD2, CD3, CD4, CD5, CD8, CD43, CD45, CD59, CD90, CD105, CD133, ABCG2, NK1.1, B220, Ter-119, Flk-2, CDCP1, Endomucin, or Gr-1. Antibodies for CD2, CD3, CD4, CD5, CD5, NK1.1, B220, Ter-119, and Gr-1 are conjugated with magnetic beads. In MACS equipment, the cells harboring CD2, CD3, CD4, CD5, CD8, NK1.1, B220, Ter-119, or Gr-1 on their surface are retained in the column equipped to trap magnetic beads and the cells attached to magnetic bead conjugated antibodies. The cells that are not captured by MACS column are subjected to FACS analysis. For FACS analysis, Antibodies for surface molecules such as c-kit, Sca-1, CD34, CD38, Thy1, are conjugated with fluorescent materials. The cells that are c-kit⁺, Sca-1⁺, CD34l^{ow/-}, CD38⁺, Thy1^{+/low} are separated from the rest of the sample by virtue of the types of fluorescent antibodies associated with the cells. These cells are provided as murine long-term HSCs suitable for use with any of the methods of the present disclosure. In other embodiments, different sets of marker are used to separate murine long-term HSCs from cells of bone marrow, umbilical cord blood, fetal tissue, and peripheral blood.

In some embodiments, obtaining HSCs from bone marrow includes first injecting the HSC donor, such as a mouse or other non-human animal, with 5-fluorouracil (5-FU) to induce the HSCs to proliferate in order to enrich for HSCs in the bone marrow of the donor.

Moreover, HSCs suitable for use with any of the methods of the present disclosure, whether obtained from, or present in, cord blood, bone marrow, peripheral blood, or other source, may be grown or expanded in any suitable, commercially available or custom defined medium (e.g., Hartshorn et al., Cell Technology for Cell Products, pages 221-224, R. Smith, Editor; Springer Netherlands, 2007). For example, serum free medium may utilize albumin and/or transferrin, which have been shown to be useful for the growth and expansion of CD34⁺ cells in serum free medium. Also, cytokines may be included, such as Flt-3 ligand, stem cell factor (SCF), and thrombopoietin (TPO), among others. HSCs may also be grown in vessels such as bioreactors (e.g., Liu et al., Journal of Biotechnology 124:592-601, 2006). A suitable medium for ex vivo expansion of HSCs may also contain HSC supporting cells, such as stromal cells (e.g., lymphoreticular stromal cells), which can be derived, for example, from the disaggregation of lymphoid tissue, and which have been shown to support the in vitro, ex vivo, and in vivo maintenance, growth, and differentiation of HSCs, as well as their progeny.

HSC growth or expansion may be measured in vitro or in vivo according to routine techniques known in the art. For example, WO 2008/073748, describes methods for measuring in vivo and in vitro expansion of HSCs, and for distinguishing between the growth/expansion of HSCs and the growth/expansion of other cells in a potentially heterogeneous population (e.g., bone marrow), including for example intermediate progenitor cells.

HSC Cell Lines

In other embodiments, HSCs suitable for use in any of the methods of the present disclosure may also be derived from an HSC cell line. Suitable HSC cell lines include any cultured hematopoietic stem cell line known in the art. Non-limiting examples include the conditionally immortalized long-term stem cell lines described in U.S. Patent Application Publication Nos. US 2007/0116691 and US 2010/0047217.

Aging Stem Cells

In certain embodiments of the methods of the present disclosure, the stem cell is an aging stem cell. Aging stem cells are typically present in older animals and may be isolated from older animals using the techniques described above. In certain embodiments, the older animal is an older human. Aging stem cells may exhibit one or more of the following properties: decreased per cell repopulating activity, decreased self-renewal and homing abilities, myeloid skewing of differentiation, and increased apoptosis with stress (Janzen et al., Nature, 2006, v. 443, p. 421). Any methods known in the art to evaluate these properties may be used to identify aging stem cells. For example, assays such as competitive repopulation and transplantation, engraftment, colony-forming unit assays, and long-term culture assays may be used.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following Examples are merely illustrative and are not meant to limit any aspects of the present disclosure in any way.

### EXAMPLES

The following examples describe the identification of a regulatory branch of the mitochondrial unfolded protein response (UPR^{mt}), which is mediated by the interplay of SIRT7 and NRF1, and is coupled to cellular energy metabolism and proliferation. SIRT7 inactivation caused reduced quiescence, increased mitochondrial protein folding stress (PFS^{mt}), and compromised regenerative capacity of hematopoietic stem cells (HSCs). SIRT7 expression was reduced in aged HSCs and SIRT7 upregulation improved the regenerative capacity of aged HSCs. These findings define the deregulation of a UPR^{mt}-mediated metabolic checkpoint as a reversible contributing factor for HSC aging and are the first evidence of a role for the UPR^{mt}-mediated metabolic checkpoint in aging.

### Example 1: SIRT7 Represses Transcription Through NRF1

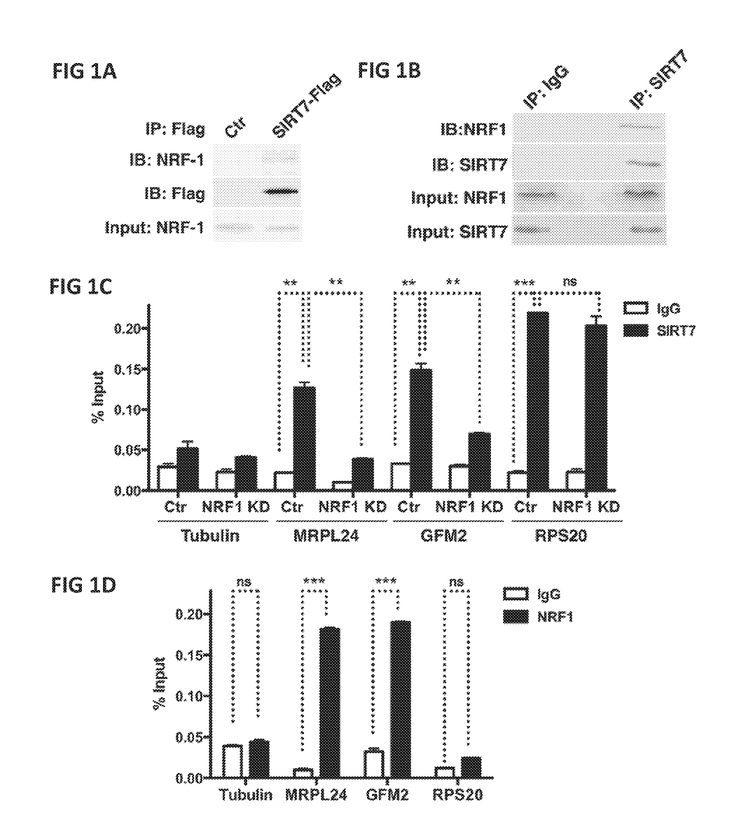

SIRT7 is a histone deacetylase that is recruited to its target promoters via interactions with transcription factors for transcriptional repression (7). A proteomic approach was taken to identify SIRT7-interacting transcription factors. 293T cells were transfected with Flag-tagged SIRT7, affinity-purified the Flag-tagged SIRT7 interactome, and identified SIRT7-interacting proteins by mass spectrometry. Among the potential SIRT7-interacting proteins was Nuclear Respiratory Factor 1 (NRF1), a master regulator of mitochondria (8). Transfected Flag-SIRT7 and endogenous SIRT7 interacted with NRF1 in 293T cells (FIGS. 1, A and B).

### Transcriptional Repression of Mitochondrial Translational Machinery

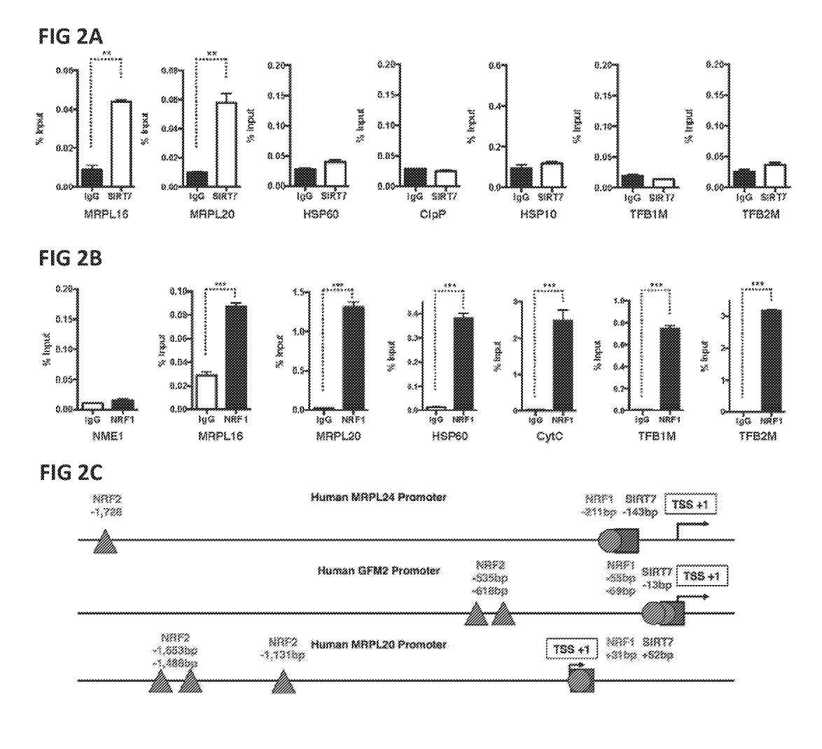

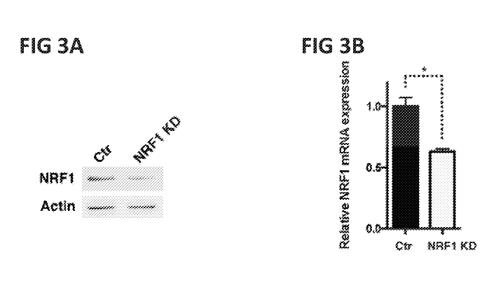

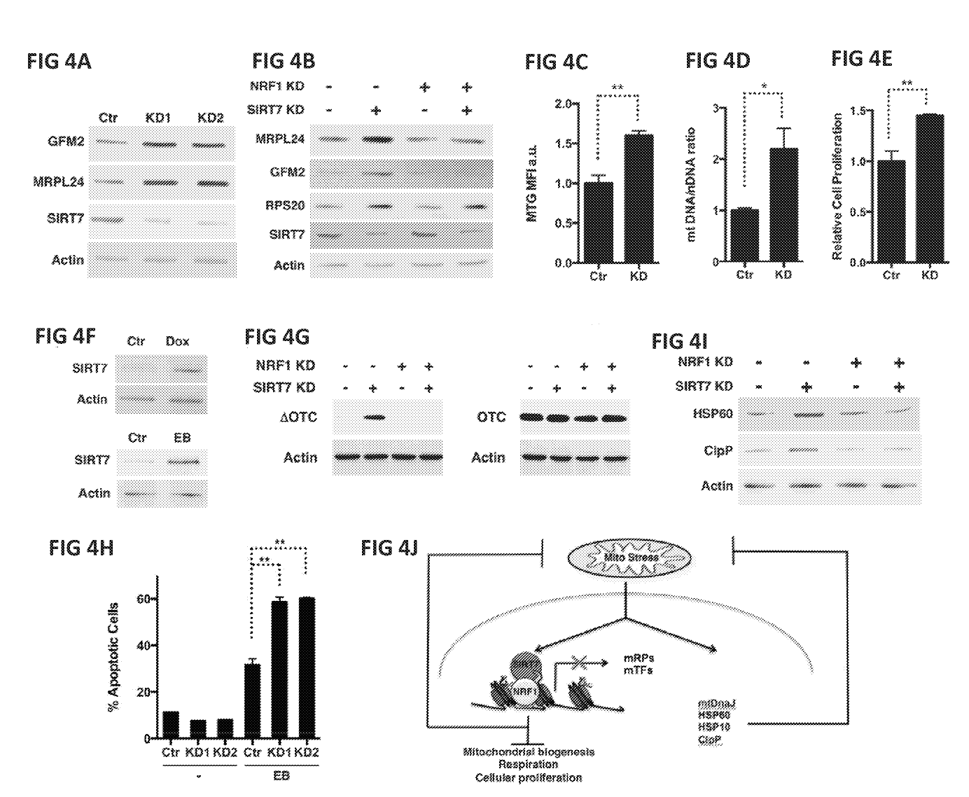

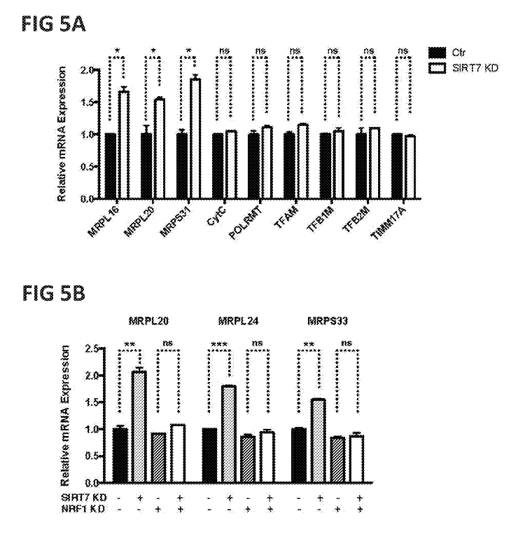

SIRT7 bound the proximal promoters of mitochondrial ribosomal proteins (mRPs) and mitochondrial translation factors (mTFs), but not other NRF1 targets (FIG. 1C and FIG. 2A and (7)). NRF1 bound the same regions as SIRT7 at the proximal promoters of mRPs and mTFs, but not RPS20 (FIG. 1D and FIG. 2B), where SIRT7 binding is mediated through Myc (9). SIRT7 binding sites were found adjacent to NRF1 consensus binding motifs at the promoters of mRPs and mTFs (FIG. 2C). NRF1 knockdown (KD) using siRNA reduced SIRT7 occupancy at the promoters of mRPs and mTFs, but not RPS20 (FIG. 1C, and FIG. 3). SIRT7 KD using short hairpin RNAs led to increased expression of mRPs and mTFs, which was abrogated by NRF1 siRNA (FIGS. 4, A and B, and FIG. 5). Thus, NRF1 targets SIRT7 specifically to the promoters of mRPs and mTFs for transcriptional repression.

### Suppression of Mitochondrial Activity and Proliferation

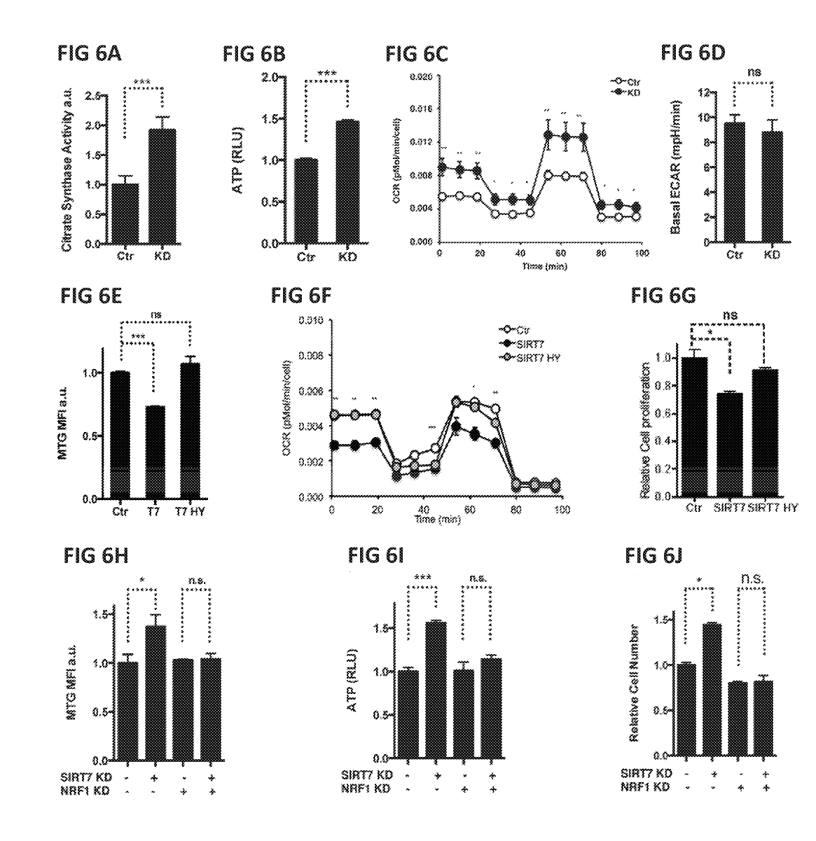

Transcriptional repression of mitochondrial and cytosolic (7, 9) translation machinery by SIRT7 suggests that SIRT7 might suppress mitochondrial activity and proliferation. SIRT7 KD cells had increased mitochondrial mass, citrate synthase activity, ATP levels, respiration, and proliferation, while cells overexpressing wild type (WT) but not a catalytically inactive SIRT7 mutant (H187Y) showed reduced mitochondrial mass, respiration, and proliferation (FIG. 4, C to E, and FIG. 6, A to G, and (10, 11)). NRF1 siRNA abrogated the increased mitochondrial activity and proliferation of SIRT7 KD cells (FIG. 6, H to J). Thus, SIRT7 represses NRF1 activity to suppress mitochondrial activity and proliferation.

### Promotion of Nutritional Stress Resistance

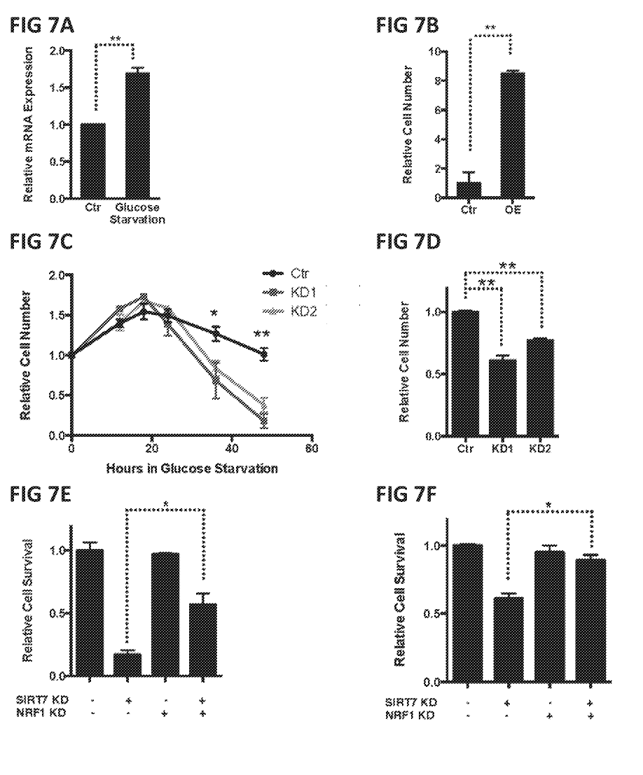

Sirtuins are increasingly recognized as stress resistance genes (12-14). Nutrient deprivation induced SIRT7 expression (FIG. 7A). Upon nutrient deprivation stress, cells reduce mitochondrial activity, growth, and proliferation to prevent cell death (15, 16). When cultured in nutrient-deprived medium, cells overexpressing SIRT7 showed increased survival, while SIRT7 KD cells showed reduced survival, which was improved by NRF1 siRNA (FIG. 7). Thus, SIRT7 suppresses NRF1 activity to promote nutritional stress resistance.

### Example 2: SIRT7 Mediates a Regulatory Branch of the UPR^{mt}

Perturbation of mitochondrial proteostasis, a form of mitochondrial stress, activates the UPR^{mt}, a retrograde signaling pathway leading to transcriptional upregulation of mitochondrial chaperones and stress relief (17, 18). Mitochondrial dysfunction results in attenuated translation, which helps restore mitochondrial homeostasis (19). SIRT7-mediated transcriptional repression of the translation machinery suggests that SIRT7 may alleviate PFS^{mt}. PFS^{mt} induced SIRT7 expression (FIG. 4F). Induction of PFS^{mt} via overexpression of an aggregation-prone mutant mitochondrial protein ornithine transcarbamylase (ΔOTC) results in UPR^{mt} activation and efficient clearance of misfolded ΔOTC (18). In SIRT7 KD cells, misfolded ΔOTC accumulated to a higher level (FIG. 4G). SIRT7 KD cells displayed increased apoptosis upon PFS^{mt} (FIG. 4H), but are not prone to general apoptosis (9). Thus, SIRT7 alleviates PFS^{mt} and promotes PFS^{mt} resistance. Consistently, mitochondrial dysfunction is manifested in the metabolic tissues of SIRT7 deficient mice (20).

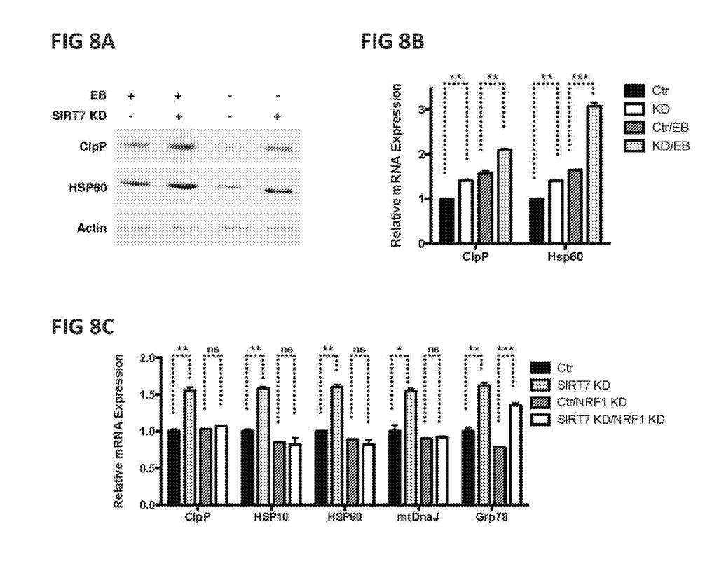

PFS^{mt} induced the expression of canonical UPR^{mt} genes in SIRT7 deficient cells (FIGS. 8, A and B), indicating that induction of SIRT7 and canonical UPR^{mt} genes are in separate branches of the UPR^{mt}. Untreated SIRT7 KD cells displayed increased expression of canonical UPR^{mt} genes (FIGS. 8, A and B), but SIRT7 did not bind to their promoters (FIG. 2A and (7)), suggesting that SIRT7 deficiency results in constitutive PFS^{mt} and compensatory induction of canonical UPR^{mt} genes. NRF1 siRNA abrogated increased PFS^{mt}, but not endoplasmic reticulum stress, in SIRT7 KD cells (FIG. 4I, and FIG. 8C). Thus, the interplay between SIRT7 and NRF1 constitutes a regulatory branch of UPR^{mt}, functioning as the nexus of reduced mitochondrial translation for homeostasis reestablishment, and repressed energy metabolism and proliferation (FIG. 4J).

### Example 3: SIRT7 is Critical for HSC Maintenance and can Reverse Aging of HSCs

### HSCs Require SIRT7 to Limit PFS^{mt}, Mitochondrial Mass, and Proliferation

What is the physiological relevance of the SIRT7-mediated UPR^{mt}? In C. elegans, the UPR^{mt} is activated during a developmental stage when a burst of mitochondrial biogenesis takes place and is attenuated when mitochondrial biogenesis subsides (17). Thus, the SIRT7-mediated UPR^{mt} may be important for cells that experience bursts of mitochondrial biogenesis and convert between growth states with markedly different bioenergetic demands and proliferative potentials, such as stem cells. Quiescent adult stem cells have low mitochondrial content, but mitochondrial biogenesis increases during proliferation and differentiation (4).

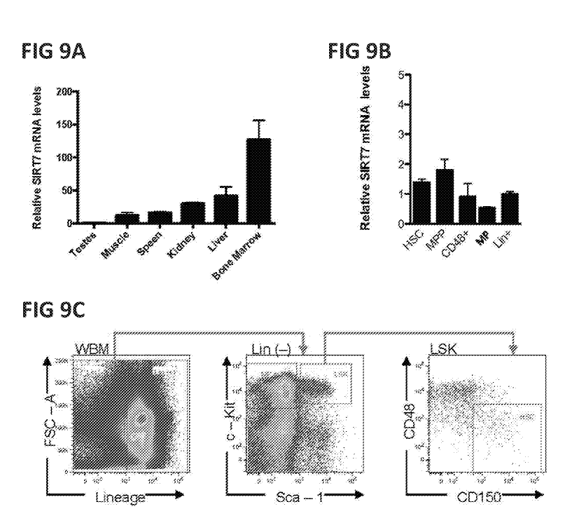

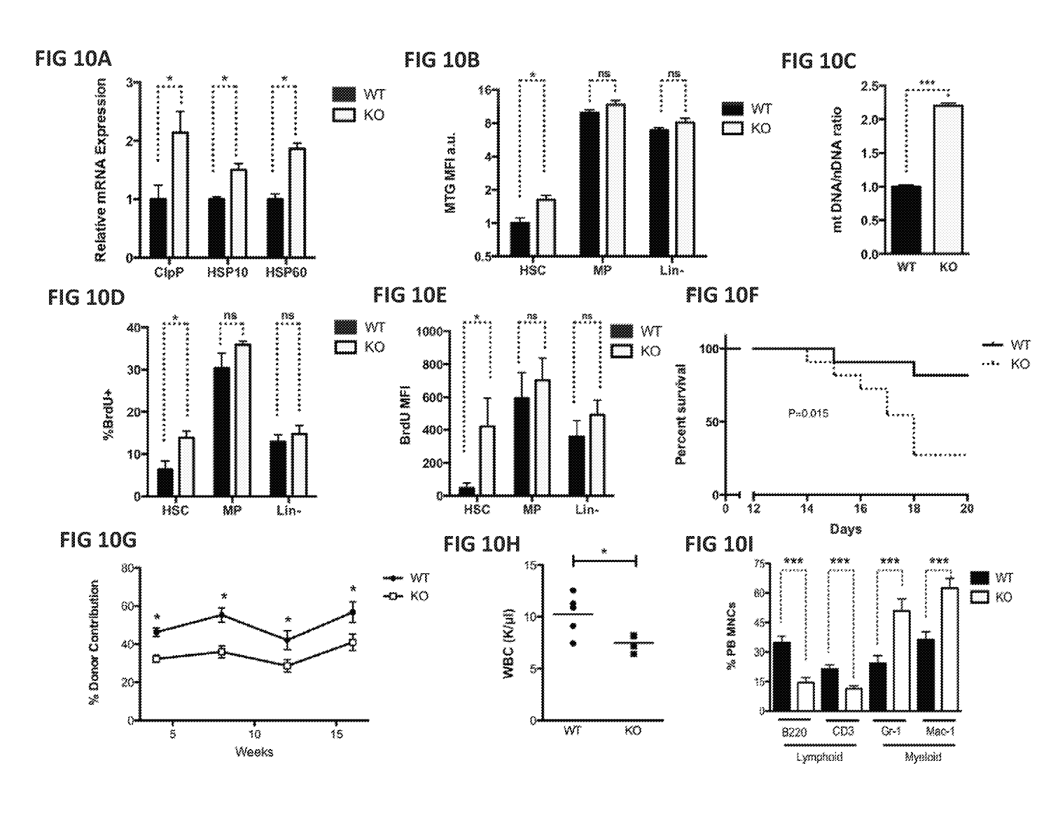

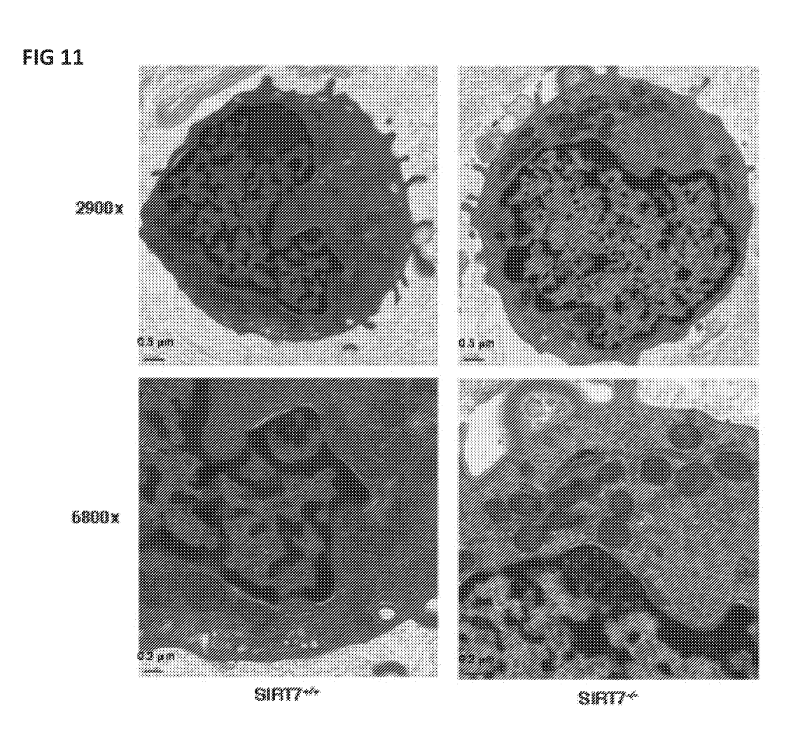

Because SIRT7 is highly expressed in the hematopoietic system, focus was given to HSCs isolated from SIRT7^{+/+} and SIRT7^{-/-} mice (FIG. 9). SIRT7^{-/-} HSCs had increased expression of UPR^{mt} genes, indicative of increased PFS^{mt} (FIG. 10A). Mitotracker Green (MTG) staining, mitochondrial DNA quantification, and electron microscopy revealed increased mitochondrial numbers in SIRT7^{-/-} HSCs (FIGS. 10, B and C, and FIG. 11). In vivo BrdU incorporation showed increased proliferation of SIRT7^{-/-} HSCs (FIG. 10D). SIRT7^{-/-} HSCs also exhibited an increased propensity to enter the cell cycle upon ex vivo culture with cytokines (FIG. 10E). However, there was no difference in mitochondrial number or proliferation in the differentiated subpopulations of these two genotypes (FIGS. 10, B, D, and E). Animals with loss of HSC quiescence are sensitive to the myeloablative drug 5-Fluorouracil (21). Upon 5-Fluorouracil treatment, mice reconstituted with SIRT7^{-/-} bone marrow cells (BMCs) died sooner than SIRT7^{+/+} controls (FIG. 10F). Thus, HSCs require SIRT7 to limit PFS^{mt}, mitochondrial mass, and proliferation.

SIRT7 Promotes HSC Maintenance, and its Downregulation in Aged HSCs Contributes to their Functional Decline

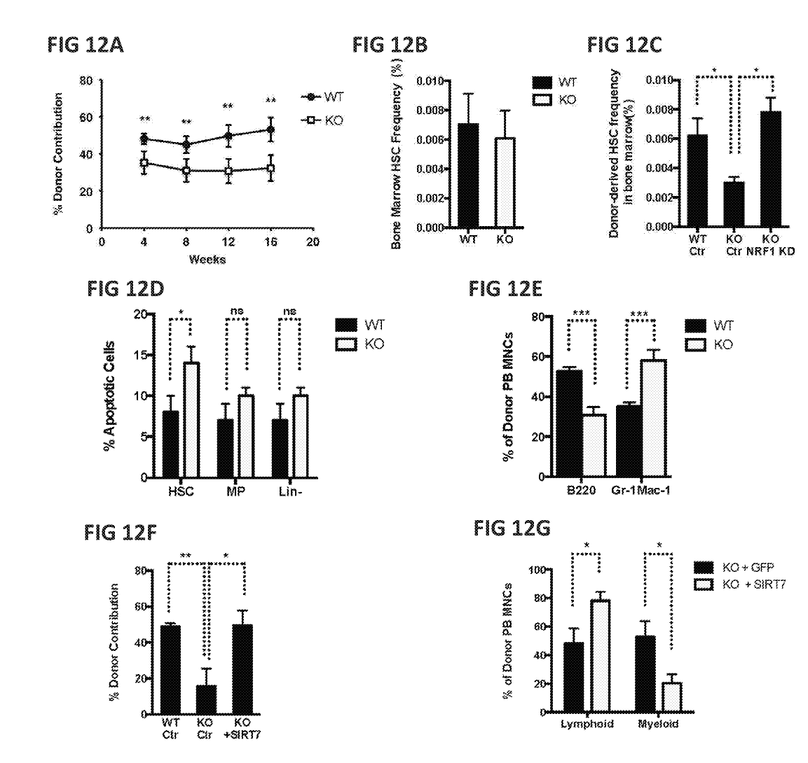

Reduced HSC quiescence causes compromised regenerative function (21). SIRT7^{-/-} BMCs or purified HSCs displayed a 40% reduction in long term reconstitution of the recipients' hematopoietic system compared to their SIRT7^{+/+} counterparts (FIG. 10G, and FIG. 12A). SIRT7^{-/-} mice also had reduced numbers of white blood cells (FIG. 10H). Although SIRT7^{+/+} and SIRT7^{-/-} mice had comparable HSC frequency in the bone marrow under steady-state conditions, there was a 50% reduction in the frequency of SIRT7^{-/-} HSCs upon transplantation (FIGS. 12, B and C). SIRT7^{-/-} HSCs showed increased apoptosis upon transplantation (FIG. 12D), which may account for compromised HSC engraftment. HSCs differentiate into lymphoid and myeloid lineages. Myeloid-biased differentiation was apparent in SIRT7^{-/-} mice or in mice reconstituted with SIRT7^{-/-} HSCs (FIG. 10I, and FIG. 12E). Thus, SIRT7 promotes HSC maintenance and prevents myeloid-biased differentiation.

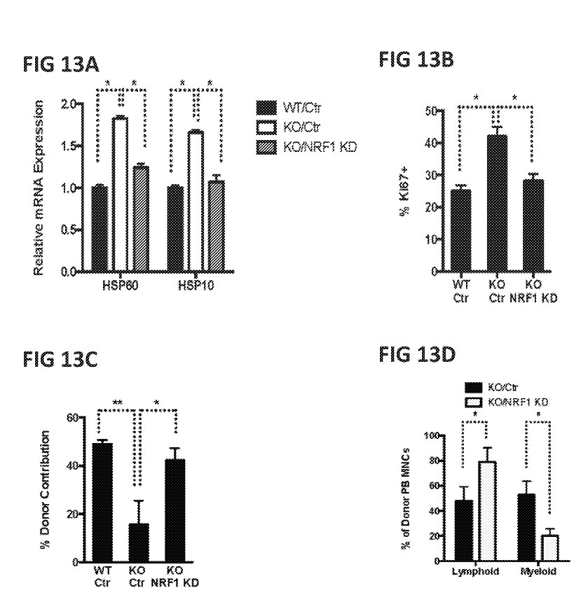

Reintroduction of SIRT7 in SIRT7^{-/-} HSCs improved reconstitution capacity and rescued myeloid-biased differentiation (FIGS. 12, F and G), indicating that SIRT7 regulates HSC maintenance cell-autonomously. NRF1 inactivation in SIRT7^{-/-} HSCs reduced PFS^{mt}, improved HSC quiescence, engraftment, reconstitution, and rescued myeloid-biased differentiation (FIGS. 12C, and 13). Thus, SIRT7 represses NRF1 activity to alleviate PFS^{mt} and ensure HSC maintenance.

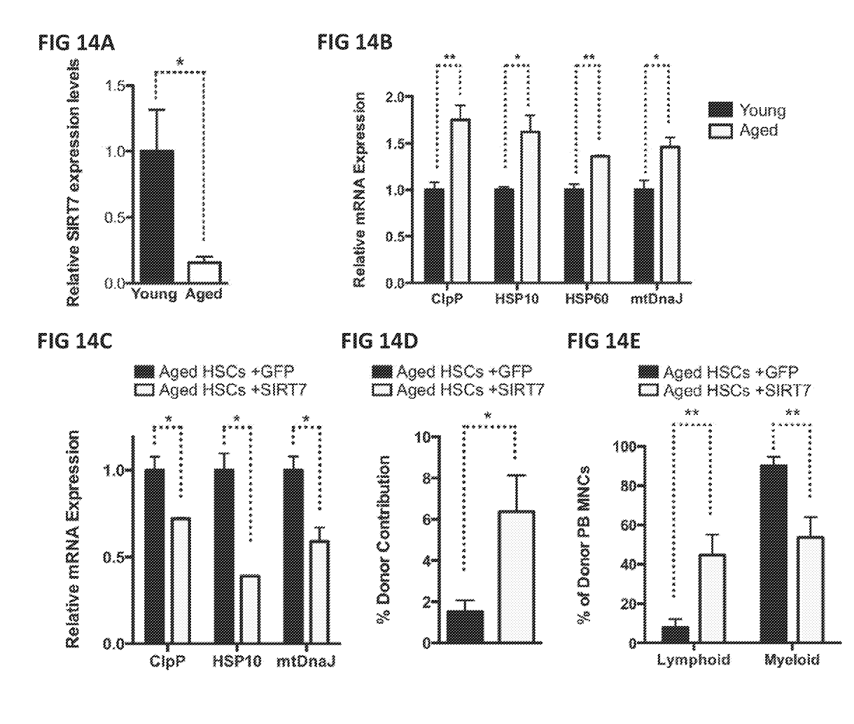

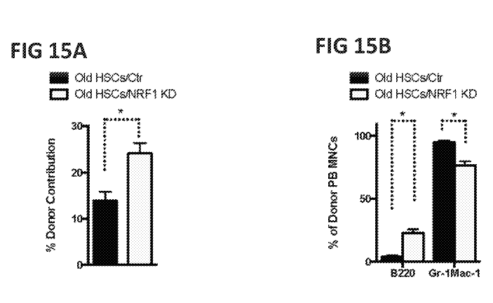

SIRT7 expression was reduced in aged HSCs (FIG. 14A and (22)). Notably, defects manifested in SIRT7^{-/-} HSCs (increased PFS^{mt} and apoptosis, loss of quiescence, decreased reconstitution capacity, and myeloid-biased differentiation) resemble aspects of aged HSCs ((23-25) and FIG. 14B). SIRT7 overexpression or NRF1 inactivation in aged HSCs reduced PFS^{mt}, improved reconstitution capacity, and rescued myeloid-biased differentiation (FIG. 14, C to E, and FIG. 15). Thus, SIRT7 downregulation results in increased PFS^{mt} in aged HSCs, contributing to their functional decline.

### Example 4: Summary of Findings

Collectively, the results highlight PFS^{mt} as a trigger of a metabolic checkpoint that regulates HSC quiescence, and establish the deregulation of UPR^{mt} as a contributing factor for HSC aging. Using a stress signal as a messenger to return to quiescence may ensure the integrity of HSCs, which persist throughout the entire lifespan for tissue maintenance. The interplay between SIRT7, which is induced upon PFS^{mt}, and NRF1, a master regulator of mitochondria, is uniquely positioned to integrate PFS^{mt} to metabolic checkpoint regulation.

SIRT7 represses NRF1 activity to reduce the expression of the mitochondrial translation machinery and to alleviate PFS^{mt} (FIGS. 1 and 4). In vivo gene expression studies cannot distinguish direct versus indirect effects. In this regard, ChIP-seq studies are informative in identifying direct SIRT7 targets (7). While gene expression changes in the metabolic tissues of SIRT7^{-/-} mice are likely reflective of severe mitochondrial and metabolic defects (20), transiently knocking down SIRT7 in cultured cells can capture the direct effect of SIRT7 on its targets ((7, 9) and FIG. 4) and may account for different gene expression changes. In contrast to the severe defects in metabolic tissues of SIRT7^{-/-} mice, SIRT7^{-/-} HSCs have increased mitochondria number and proliferation under homeostatic conditions, but do not fare well upon transplantation stress (FIG. 10). These observations are consistent with the notion that while metabolic tissues have a large number of mitochondria, HSCs have very few mitochondria under homeostatic conditions and increase mitochondrial biogenesis upon transplantation. The combined power of biochemistry, cell culture, and mouse genetics is necessary to tease out direct and indirect effects of SIRT7 under various physiological conditions. The proposed model (FIG. 4J) is consistent with the functions of SIRT7 in chromatin remodeling and gene repression (7), stress responses ((9) and FIG. 4), and mitochondrial maintenance ((20) and FIG. 4).

Reintroduction of SIRT7 in aged HSCs reduces PFS^{mt} and improves their regenerative capacity. Thus, PFS^{mt}-induced HSC aging is reversible. It appears that HSC aging is not due to passive chronic accumulation of cellular damage over the lifetime, but the regulated repression of cellular protective programs. The dysregulated UPR^{mt} cellular protective program may be targeted to reverse HSC aging and rejuvenate tissue homeostasis.

### Example 5: Materials and Methods

The following sections describe the materials and methods used in the experiments described in Examples 1-4.

### Cell Culture and RNAi

293T cells were acquired from the ATCC. Cells were cultured in advanced DMEM (Invitrogen) supplemented with 1% penicillin-streptomycin (Invitrogen) and 10% FBS (Invitrogen). For inducing PFS^{mt}, cells were treated with doxycycline (30 μg/mL) for 48 hrs, or ethidium bromide (50 ng/mL) for 7 days. Alternatively, cells were transfected with a construct expressing an aggregation prone mutant mitochondrial OTC protein from the Hoogenraad lab (18). For nutrient deprivation, cells were cultured in glucose free medium (Invitrogen) or glutamine free medium (Invitrogen) for 48-68 hours. Cell proliferation and survival were scored using a Vi-Cell Analyzer (Beckman Coulter).

SIRT7 knockdown target sequences are as follows, as previously described (9):

S7KD1,
(SEQ ID NO: 1)
5′-CACCTTTCTGTGAGAACGGAA-3′;
S7KD2,
(SEQ ID NO: 2)
5′-TAGCCATTTGTCCTTGAGGAA-3′,

NRF1 knockdown target sequences are as follows:

NRF1 KD (mouse),
(SEQ ID NO: 3)
5′-GAAAGCTGCAAGCCTATCT-3′
NRF1 KD (human),
(SEQ ID NO: 4)
5′-CACCGTTGCCCAAGTGAATTA-3′

Double-stranded siRNAs were purchased from Thermo Scientific and were transfected into cells via RNAiMax (Invitrogen) according to manufacturer's instructions. Generation of SIRT7 knockdown and overexpressing cells was described previously (9). After puromycin selection, cells were recovered in puromycin free medium for 2-3 passages before analyses.

### Measurements of Mitochondrial Mass, ATP, Citrate Synthase Activity, and Oxygen Consumption

To measure mitochondrial mass, cells were stained with 100 nM MitoTracker Green (Invitrogen) for 30 minutes in at 37° C., and analyzed with flow cytometry (BD Fortessa).

For oxygen consumption, 3×105 cells were plated using CellTak (BD) and the oxygen consumption rate (OCR) was measured using a Seahorse XF24 instrument following the manufacturer's instructions (Seahorse Biosciences). OCR was measured under basal conditions, in the presence of the mitochondrial inhibitor oligomycin A (1 μM), mitochondrial uncoupler FCCP (1 μM) and respiratory chain inhibitor antimycin and rotenone (1 μM).

Citrate synthase activity was measured following the manufacturer's instruction (Biovision Citrate Synthase Activity Colorimetric Assay Kit #K318-100). To measure ATP, cells in suspension were mixed with an equal volume of CellTiterGlo in solid white luminescence plates (Grenier Bio-One) following the manufacturer's instructions (Promega). Luminescence was measured using a luminometer (LMAX II 384 microplate reader, Molecular Devices) to obtain relative luciferase units (RLU).

### Co-Immunoprecipitations

Co-immunoprecipitations were performed as previously described (14) with Flag-resin (Sigma) or Protein A/G beads (Santa Cruz) for SIRT7 IP. Elution was performed with either Flag peptide (Sigma) or 100 mM Glycine solution (pH 3) for SIRT7 IP. Antibodies are provided in Table 1, below.

TABLE 1
Antibodies used.

| Antibodies | Source | Catalog # |
|---|---|---|
| SIRT7 | Abnova | H00051547 |
| Beta Actin | Sigma | A2066 |
| Flag | Sigma | F1804 |
| IgG | Santa Cruz | SC-2027 |
| RP520 | Abcam | Ab74700 |
| NRF-1 | Proteintech | 12482-1-AP |
| MRPL24 | Proteintech | 16224-1-AP |
| GFM2 | Proteintech | 16941-1-AP |
| ClpP | Proteintech | 15698-1-AP |
| HSP60 | Cell Signaling | 12165S |
| OTC | Santa Cruz | SC-102051 |

| FACS Antibodies & | | | |
|---|---|---|---|
| Reagents | Source | Catalog # | Clone # |
| CD45.1 PerCP | Biolegend | 110726 | A20 |
| Streptavidin PerCP | Biolegend | 405213 | |
| Mac1 PerCP | Biolegend | 101230 | M1/70 |
| CD3 Pacific Blue | Biolegend | 100214 | 17A2 |
| Sca1 Pacific Blue | Biolegend | 108120 | D7 |
| Streptavidin APC-Cy7 | Biolegend | 405208 | |
| c-Kit APC-Cy7 | Biolegend | 105826 | 2B8 |
| CD45.2 Cy7-PE | Biolegend | 109830 | 104 |
| CD150 Cy7-PE | Biolegend | 115914 | TC15-12F12.2 |
| Gr1 Cy7-PE | Biolegend | 108416 | RB6-8C5 |
| c-Kit Cy7-PE | Biolegend | 105813 | 2B8 |
| Streptavidin Cy7-PE | Biolegend | 405206 | |
| CD3 Biotin | Biolegend | 100304 | 145-2C11 |
| B220 Biotin | Biolegend | 103204 | RA3-6B2 |
| Gr1 Biotin | Biolegend | 108404 | RB6-8C5 |
| CD8a Biotin | Biolegend | 100704 | 53-6.8 |
| Mac1 Biotin | Biolegend | 101204 | M1/70 |
| Ter119 Biotin | Biolegend | 116204 | TER-119 |
| CD4 Biotin | Biolegend | 100404 | GK1.5 |
| CD48 FITC | Biolegend | 103404 | HM48-1 |
| Gr1 FITC | Biolegend | 108406 | RB6-8C5 |
| CD45.2 FITC | eBioscience | 11-0454-85 | 104 |
| AnnexinV FITC | Biolegend | 640906 | |
| Ki67 A488 | Biolegend | 350508 | Ki-67 |
| CD150 PE | Biolegend | 115904 | TC15-12F12.2 |
| CD45.1 PE | Biolegend | 110708 | A20 |
| Mac1 | Biolegend | 101208 | M1/70 |
| c-Kit APC | Biolegend | 105812 | 2B8 |
| B220 APC | Biolegend | 103212 | RA3-6B2 |
| Ki67 APC | Biolegend | 350514 | Ki-67 |
| AnnexinV APC | Biolegend | 640920 | |
| CD48 A647 | Biolegend | 103416 | HM48-1 |
| Fixation buffer | Biolegend | 420801 | |
| Permeabilization wash | Biolegend | 421002 | |
| buffer | | | |
| AnnexinV binding buffer | Biolegend | 422201 | |
| 7AAD | Biolegend | 420404 | |
| BrdU labeling reagent | Invitrogen | 000103 | |

ChIP and mRNA Analysis

Cells were prepared for ChIP as previously described (26), with the exception that DNA was washed and eluted using a PCR purification kit (Qiagen) rather than by phenolchloroform extraction. RNA was isolated from cells or tissues using Trizol reagent (Invitrogen) and purified using the RNeasy Mini Kit (Qiagen). cDNA was generated using the qScript™ cDNA SuperMix (Quanta Biosciences). Gene expression was determined by real time PCR using Eva qPCR SuperMix kit (BioChain Institute) on an ABI StepOnePlus system. All data were normalized to ActB or GAPDH expression. Antibodies and PCR primer details are provided in Tables 1-3.

TABLE 2
Primers used for qPCR analysis.

| Gene | Primer | Sequence | SEQ ID NO: |
|---|---|---|---|
| SIRT7 (human) | Forward | CGCCAAATACTTGGTCGTCT | 5 |
| | Reverse | CCCTTTCTGAAGCAGTGTCC | 6 |
| ClpP (human) | Forward | CTCTTCCTGCAATCCGAGAG | 7 |
| | Reverse | GGATGTACTGCATCGTGTCG | 8 |
| Hsp10 (human) | Forward | CAGTAGTCGCTGTTGGATCG | 9 |
| | Reverse | TGCCTCCATATTCTGGGAGA | 10 |
| Hsp60 (human) | Forward | TGACCCAACAAAGGTTGTGA | 11 |
| | Reverse | CATACCACCTCCCATTCCAC | 12 |
| mtDnaJ (human) | Forward | CGAAATGGCAGAAGAAGAGG | 13 |
| | Reverse | TGCATGCACTACAGAGCACA | 14 |
| Grp78 (human) | Forward | TCATCGGACGCACTTGGAA | 15 |
| | Reverse | CAACCACCTTGAATGGCAAGA | 16 |
| ClpP (mouse) | Forward | CTGCCCAATTCCAGAATCAT | 17 |
| | Reverse | TGTAGGCTCTGCTTGGTGTG | 18 |
| Hsp10 (mouse) | Forward | CCAAAGGTGGCATTATGCTT | 19 |
| | Reverse | TGACAGGCTCAATCTCTCCA | 20 |
| Hsp60 (mouse) | Forward | ACCTGTGACAACCCCTGAAG | 21 |
| | Reverse | TGACACCCTTTCTTCCAACC | 22 |
| mtDnaJ (mouse) | Forward | GAGCTGAAGAAGGCATACCG | 23 |
| | Reverse | CAGCTCTCGCTTCTCTGGAT | 24 |
| ND4 (mtDNA) (mouse) | Forward | GGAACCAAACTGAACGCCTA | 25 |
| | Reverse | ATGAGGGCAATTAGCAGTGG | 26 |
| b2 microglobulin | Forward | TCATTAGGGAGGAGCCAATG | 27 |
| (nDNA) (Mouse) | | | |
| | Reverse | ATCCCCTTTCGTTTTTGCTT | 28 |
| SIRT7 (mouse) | Forward | CCATGGGAAGTGTGATGATG | 29 |
| | Reverse | TCCTACTGTGGCTGCCTTCT | 30 |
| MRPL16 | Forward | ACATACGGGGACCTTCCACT | 31 |
| | Reverse | AAACATGTTCTTGGGGTCCA | 32 |
| MRPL20 | Forward | GAACATGAGGACCCTCTGGA | 33 |
| | Reverse | CCGCTAGGACTTTCCTGTTG | 34 |
| MRPL24 | Forward | GGGGAACCATGATCCCTAGT | 35 |
| | Reverse | AATTCTCCCTGATCGTGTGG | 36 |
| MRPS31 | Forward | GAGGAAGAGTCAAGGGCACA | 37 |
| | Reverse | CTGAATCCGAAGCTCTGGTC | 38 |
| MRPS33 | Forward | ATATGCCTTCCGCATGTCTC | 39 |
| | Reverse | GCCAAGGGCAGTTCACTAAA | 40 |
| CytC | Forward | AAGTGTTCCCAGTGCCACA | 41 |
| | Reverse | GTTCTTATTGGCGGCTGTGT | 42 |
| POLRMT | Forward | AAAGCCCAACACACGTAAGC | 43 |
| | Reverse | GTGCACAGAGACGAAGGTCA | 44 |
| TFAM | Forward | TGGCAAGTTGTCCAAAGAAA | 45 |
| | Reverse | ACGCTGGGCAATTCTTCTAA | 46 |
| TFB1M | Forward | CTCCCTTGATACAGCCCAAG | 47 |
| | Reverse | TGCGCTTCAGGGAATAACAT | 48 |
| TFB2M | Forward | AGATCCCGGAAATCCAGACT | 49 |
| | Reverse | CTACGCTTTGGGTTTTCCAG | 50 |
| TIMM17A | Forward | AGGGCTGTTTTCCATGATTG | 51 |
| | Reverse | CCACTGGTCCATTTCTTGCT | 52 |
| NRF1 | Forward | CCCAGGCTCAGCTTCGGGCA | 53 |
| | Reverse | GCTCTTCTGTGCGGACATCAC | 54 |
| tRNALeu (mtDNA) | Forward | CACCCAAGAACAGGGTTTGT | 55 |
| (human) | Reverse | TGGCCATGGGTATGTTGTTA | 56 |
| β2-microglobulin | Forward | TGCTGTCTCCATGTTTGATGTATCT | 57 |
| (nDNA) (human) | Reverse | TCTCTGCTCCCCACCTCTAAGT | 58 |
| 16S rRNA (mtDNA) | Forward | GCCTTCCCCCGTAAATGATA | 59 |
| (human) | Reverse | TTATGCGATTACCGGGCTCT | 60 |

TABLE 3
PCR primers used for ChIP analysis

| Target | Primer | Sequence | SEQ ID NO: |
|---|---|---|---|
| γ-tubulin | Forward | ACGGGTTTCATCATGTTTGTT | 61 |
| | Reverse | GGCAGATCCCCTGAGGTC | 62 |
| RPS20 | Forward | AAGTTCTTTCTTTTTGAGGAAGACG | 63 |
| | Reverse | GAACAGCGGTGAGTCAGGA | 64 |
| GFM2 | Forward | CGGGACAGGAAAGAGTCACC | 65 |
| | Reverse | CGGAAAACAGAGGCTCGGAA | 66 |
| mRPL24 | Forward | TGAACAGGAAGCCACAACA | 67 |
| | Reverse | GAGGCCGCTGGGAATTGTAG | 68 |
| NME1 | Forward | CCGTAATACTTGGCTCTCGAA | 69 |
| | Reverse | GAATAGACCTGCATGAAGTGAGG | 70 |
| HSP60 | Forward | CAGCGACTACTGTTGCTTGC | 71 |
| | Reverse | ACAGGCAGGACAAGCGTTTA | 72 |
| TFB1M | Forward | CCTAGTCCACCCGGCTCT | 73 |
| | Reverse | GAGGAACCTGCGAGACCTAA | 74 |
| TFB2M | Forward | ACGGTCCACTCACAATCCTC | 75 |
| | Reverse | CCCACGTGGAACATTTTCTG | 76 |
| Cytc | Forward | CCGTACACCCTAACATGCTC | 77 |
| | Reverse | TGGCACAACGAACACTCC | 78 |
| mRPL16 | Forward | TCTTCTGGGGAAAGACTGGA | 79 |
| | Reverse | TGAGTTCCTGCGGTCAAAG | 80 |
| mRPL20 | Forward | CGAGTTCAGGAGCACAACTG | 81 |
| | Reverse | GTCAGCCCCTGCGATACTT | 82 |
| ClpP | Forward | ATGTGGCCCGGAATATTGGT | 83 |
| | Reverse | CAGGCCGTTCTGGAGTGTC | 84 |
| HSP10 | Forward | AGAGGAGGAAGGCCCTC | 85 |
| | Reverse | CTGCACTCTGTCCCTCACTC | 86 |

### Mice

SIRT7^{-/-} mice have been described previously (9). All mice were housed on a 12:12 hr light:dark cycle at 25° C. For 5-Fluorouricil treatment study, 1×106 BMCs from SIRT7^{+/+} or SIRT7^{-/-} mice were transplanted into lethally irradiated recipient mice. Four months posttransplantation, 5-Fluorouricil was administrated to mice intraperitoneally at a dose of 150 mg/kg once per week, and the survival of the mice was monitored daily. All animal procedures were in accordance with the animal care committee at the University of California, Berkeley.

### Flow Cytometry and Cell Sorting

BMCs were obtained by crushing the long bones with sterile PBS without calcium and magnesium supplemented with 2% FBS. Lineage staining contained a cocktail of biotinylated anti-mouse antibodies to Mac-1 (CD11b), Gr-1 (Ly-6G/C), Ter119 (Ly-76), CD3, CD4, CD8a (Ly-2), and B220 (CD45R) (BioLegend). For detection or sorting, we used streptavidin conjugated to APC-Cy7, c-Kit-APC, Sca-1-Pacific blue, CD48-FITC, and CD150-PE (BioLegend). For congenic strain discrimination, anti-CD45.1 PerCP and anti-CD45.2 PE-Cy7 antibodies (BioLegend) were used. For assessment of apoptosis and cell cycle analysis, AnnexinV and Ki-67 (BioLegend) staining were performed respectively according to the manufacturer's recommendation after cell surface staining. For in vivo cell-cycle analysis, BrdU (Invitrogen) was incorporated over a 16-hour period. For ex vivo proliferation analysis, cultured BMCs were pulsed with BrdU (Invitrogen) for one hour before flow cytometry analysis. For mitochondrial mass, BMCs were incubated with 100 nM MitoTracker Green (Invitrogen) for 30 min at 37° C. in the dark after cell surface staining. All data were collected on a Fortessa (Becton Dickinson), and data analysis was performed with FlowJo (TreeStar). For cell sorting, lineage depletion or c-kit enrichment was performed according to the manufacturer's instructions (Miltenyi Biotec). Cells were sorted using a Cytopeia INFLUX Sorter (Becton Dickinson). Antibody details are provided in Table 1.

### Lentiviral Transduction of HSCs

As previously described (27), sorted HSCs were prestimulated for 5-10 hrs in a 96 well U bottom dish in StemSpan SFEM (Stem Cell Technologies) supplemented with 10% FBS (Stem Cell Technologies), 1% Penicillin/Streptomycin (Invitrogen), IL3 (20 ng/ml), IL6 (20 ng/ml), TPO (50 ng/ml), Flt3L (50 ng/ml), and SCF (100 ng/ml) (Peprotech).

SIRT7 was cloned into the pFUGw lentiviral construct. NRF1 shRNA was cloned into pFUGw-H1 lentiviral construct. Lentivirus was produced as described (14), concentrated by centrifugation, and resuspended with supplemented StemSpan SFEM media. The lentiviral media were added to HSCs in a 24 well plate, spinoculated for 90 min at 270 g in the presence of 8 ug/ml polybrene. This process was repeated 24 hr later with a fresh batch of lentiviral media.

mtDNA/nDNA

The mitochondrial DNA/nuclear DNA (mtDNA/nDNA) ratio was determined by isolating DNA from cells with Trizol (Invitrogen), as described previously (28). The ratio of mtDNA/nDNA was calculated as previously described (29).

### Electron Microscopy

40,000 HSCs were pelleted at 150 g. Samples were fixed with 2% gluaraldehyde for 10 minutes at room temperature while rocking. Samples were pelleted at 600 g and further fixed with 2% glutaraldehyde/0.1M NaCacodylate at 4° C. and were submitted to the UC Berkeley Electron Microscope Core Facility for standard transmission electron microscopy ultrastructure analyses.

### Transplantation Assays

For transplantations, 5×10⁵ BMCs from SIRT7^{+/+} or SIRT7^{-/-} CD45.2 littermates was mixed with 5×10⁵ CD45.1 B6.SJL (Jackson Laboratory) competitor BMCs and injected into lethally irradiated (950 Gy) CD45.1 B6.SJL recipient mice. Alternatively, 250 sorted HSCs from SIRT7^{+/+} or SIRT7^{-/-} mice were mixed with 5×10⁵ CD45.1 B6.SJL competitor BMCs and injected into lethally irradiated B6.SJL recipient mice. To assess multilineage reconstitution of transplanted mice, peripheral blood was collected every month for 4 months by retroorbital bleeding. Red blood cells were lysed and the remaining blood cells were stained with CD45.2 FITC, CD45.1 PE, Mac1 PerCP, Gr1 Cy7PE, B220 APC, and CD3 PB (Biolegend). Antibody details are provided in Table 1.

### Statistical Analysis

The number of mice chosen for each experiment is based on the principle that the minimal number of mice is used to have sufficient statistical power and is comparable to published literature for the same assays performed. Mice were randomized to groups and analysis of mice and tissue samples were performed by investigators blinded to the treatment of genetic background of the animals. Transplant experiments have been repeated 5 times. HSC characterizations in SIRT7^{-/-} mice have been repeated in 10 different cohorts of mice. Analyses in SIRT7 KD cells have been repeated 2-5 times. Experiments are repeated by at least 2 different scientists. Statistical analysis was performed with Excel (Microsoft) and Prism 5.0 Software (GraphPad Software). Means between two groups were compared with two-tailed, unpaired Student's t-test. Error Bars represent standard errors. In all corresponding Figures, * represents p<0.05, ** represents p<0.01, *** represents p<0.001, and ns represents p>0.05.

### REFERENCES

1. C. J. Kenyon, The genetics of ageing. Nature 464, 504 (Mar. 25, 2010).
2. C. Lopez-Otin, M. A. Blasco, L. Partridge, M. Serrano, G. Kroemer, The hallmarks of aging. Cell 153, 1194 (Jun. 6, 2013).
3. J. Vijg, J. Campisi, Puzzles, promises and a cure for ageing. Nature 454, 1065 (Aug. 28, 2008).
4. C. D. Folmes, P. P. Dzeja, T. J. Nelson, A. Terzic, Metabolic plasticity in stem cell homeostasis and differentiation. Cell stem cell 11, 596 (Nov. 2, 2012).
5. L. Liu et al., Chromatin modifications as determinants of muscle stem cell quiescence and chronological aging. Cell reports 4, 189 (Jul. 11, 2013).
6. A. E. Webb et al., FOXO3 shares common targets with ASCL1 genome-wide and inhibits ASCL1-dependent neurogenesis. Cell reports 4, 477 (Aug. 15, 2013).
7. M. F. Barber et al., SIRT7 links H3K18 deacetylation to maintenance of oncogenic transformation. Nature 487, 114 (Jul. 5, 2012).
8. R. C. Scarpulla, Transcriptional paradigms in mammalian mitochondrial biogenesis and function. Physiological reviews 88, 611 (April, 2008).
9. J. Shin et al., SIRT7 Represses Myc Activity to Suppress ER Stress and Prevent Fatty Liver Disease. Cell reports 5, 654 (Nov. 14, 2013).
10. J. Y. Lu, Y. Zhang, Y. F. Shen, [Inhibitory role of SirT7 in the growth of P19 cell line]. Zhongguo yi xue ke xue yuan xue bao. Acta Academiae Medicinae Sinicae 31, 724 (December, 2009).
11. O. Vakhrusheva, D. Braeuer, Z. Liu, T. Braun, E. Bober, Sirt7-dependent inhibition of cell growth and proliferation might be instrumental to mediate tissue integrity during aging. Journal of physiology and pharmacology: an official journal of the Polish Physiological Society 59 Suppl 9, 201 (December, 2008).
12. K. Brown et al., SIRT3 reverses aging-associated degeneration. Cell reports 3, 319 (Feb. 21, 2013).
13. W. Giblin, M. E. Skinner, D. B. Lombard, Sirtuins: guardians of mammalian healthspan. Trends in genetics: TIG 30, 271 (July, 2014).
14. X. Qiu, K. Brown, M. D. Hirschey, E. Verdin, D. Chen, Calorie restriction reduces oxidative stress by SIRT3-mediated SOD2 activation. Cell metabolism 12, 662 (Dec. 1, 2010).
15. K. Inoki, T. Zhu, K. L. Guan, TSC2 mediates cellular energy response to control cell growth and survival. Cell 115, 577 (Nov. 26, 2003).
16. M. Laplante, D. M. Sabatini, mTOR signaling in growth control and disease. Cell 149, 274 (Apr. 13, 2012).
17. C. M. Haynes, D. Ron, The mitochondrial UPR-protecting organelle protein homeostasis. Journal of cell science 123, 3849 (Nov. 15, 2010).
18. Q. Zhao et al., A mitochondrial specific stress response in mammalian cells. The EMBO journal 21, 4411 (Sep. 2, 2002).
19. C. M. Haynes, C. J. Fiorese, Y. F. Lin, Evaluating and responding to mitochondrial dysfunction: the mitochondrial unfolded-protein response and beyond. Trends in cell biology 23, 311 (July, 2013).
20. D. Ryu et al., A SIRT7-Dependent Acetylation Switch of GABPbetal Controls Mitochondrial Function. Cell metabolism 20, 856 (Sep. 3, 2014).
21. K. Miyamoto et al., Foxo3a is essential for maintenance of the hematopoietic stem cell pool. Cell stem cell 1, 101 (Jun. 7, 2007).
22. S. M. Chambers et al., Aging hematopoietic stem cells decline in function and exhibit epigenetic dysregulation. PLoS biology 5, e201 (August, 2007).
23. V. Janzen et al., Stem-cell ageing modified by the cyclin-dependent kinase inhibitor p16INK4a. Nature 443, 421 (Sep. 28, 2006).
24. J. Oh, Y. D. Lee, A. J. Wagers, Stem cell aging: mechanisms, regulators and therapeutic opportunities. Nature medicine 20, 870 (August, 2014).
25. R. A. Signer, S. J. Morrison, Mechanisms that regulate stem cell aging and life span. Cell stem cell 12, 152 (Feb. 7, 2013).
26. J. A. Dahl, P. Collas, A quick and quantitative chromatin immunoprecipitation assay for small cell samples. Frontiers in bioscience: a journal and virtual library 12, 4925 (2007).
27. C. Zhao et al., Hedgehog signalling is essential for maintenance of cancer stem cells in myeloid leukaemia. Nature 458, 776 (Apr. 9, 2009).
28. L. Lai et al., Transcriptional coactivators PGC-1alpha and PGC-1beta control overlapping programs required for perinatal maturation of the heart. Genes & development 22, 1948 (Jul. 15, 2008).
29. V. Venegas, J. Wang, D. Dimmock, L. J. Wong, Real-time quantitative PCR analysis of mitochondrial DNA content. Curr Protoc Hum Genet Chapter 19:Unit 19.7. doi: 10.1002/0471142905.hg1907s68. (2011).

All references cited herein, including patent applications and publications, are hereby incorporated by reference in their entirety.

1. A method of reversing aging of stem cells comprising activating the mitochondrial unfolded protein response in a stem cell, wherein aging of the stem cell is reversed.

2. The method of claim 1 wherein the mitochondrial unfolded protein response is activated by activating SIRT7 in the stem cell.

3. The method of claim 1 wherein the mitochondrial unfolded protein response is activated by activating a mitochondrial stress protein.

4. The method of claim 3 wherein the mitochondrial stress protein is selected from the group consisting of mtDnaJ, HSP60, HSP10, and ClpP in the stem cell.

5. The method of claim 2 wherein SIRT7 is activated by increasing the transcription of the sirt7 gene in the stem cell.

6. The method of claim 2 wherein SIRT7 is activated by increasing the translation of SIRT7 protein in the stem cell.

7. The method of claim 2 wherein SIRT7 is activated by delivering an exogenous copy of the sirt7 gene to the stem cell wherein the exogenous sirt7 gene is expressed in the stem cell.

8. The method of claim 7 wherein the exogenous copy of sirt7 gene is delivered to the stem cell with a viral vector.

9. The method of claim 2, wherein the SIRT7 is activated by a small molecule.

10. The method of claim 2, wherein the SIRT7 is activated by increasing intracellular NAD levels.

11. The method of claim 10 wherein intracellular NAD levels are increased by delivering small molecules that activate NAD synthesis enzymes to the stem cell.

12. The method of claim 10 wherein intracellular NAD levels are increased by increasing the level of a NAD precursor in the stem cell.

13. The method of claim 12 wherein the NAD precursor is selected from the group consisting of nicotinamide mono nucleotide (NMN) and nicotinamide riboside (NR).

14. The method of claim 10 wherein intracellular NAD levels are increased by increasing the level of a NAD biosynthesis enzyme in the stem cell.

15. The method of claim 1 wherein the stem cell exhibits one or more of the characteristics selected from the group consisting of reduced occurrence of cell death, increased quiescence, increased occurrence of self-renewal, increased ability to repopulate its organ or tissue of origin, increased homing ability, and a change in differentiation profile.

16. The method of claim 1 wherein the stem cell is in an animal.

17. The method of claim 16 wherein the animal is a human.

18. The method of claim 1, wherein the stem cell is a hematopoietic stem cell.

19. The method of claim 18 wherein the hematopoietic stem cell exhibits improved performance in an assay selected from the group consisting of HSC engraftment, bone marrow reconstitution, and competitive transplantation.

20. The method of claim 17 wherein blood drawn from the human does not exhibit one or more characteristics selected from the group consisting of increased myeloid differentiation, fewer lymphoid cells, and anemia.

21. The method of claim 1 wherein the stem cell originated in an organ or tissue selected from the group consisting of brain, spinal cord, peripheral blood, blood vessels, skeletal muscle, skin, teeth, hair follicle, heart, gut, liver, ovarian epithelium, and testis.

22. The method of claim 1, wherein the stem cell is an aging stem cell.

23. A method for preventing aging of stem cells comprising activating the mitochondrial unfolded protein response in a stem cell, wherein aging of the stem cell is prevented.

24. The method of claim 23 wherein aging of the stem cell is delayed.

25. A method for promoting stem cell maintenance comprising activating the mitochondrial unfolded protein response in a stem cell, wherein the stem cell continues to self-renew.

26. A method for preventing and/or reversing tissue degeneration or injury comprising activating the mitochondrial unfolded protein response in a stem cell, wherein the stem cell is in an animal and wherein degeneration or injury of a tissue in the animal is prevented and/or reversed.
